(19) United States
(12) Patent Application Publication
(10) Pub. No.: US 2005/0069999 A1
(43) Pub. Date: Mar. 31, 2005

(54) Sh3 domain binding inhibitors

(76) Inventors: Sreenath V Sharma, Sayre, PA (US); Noriko Matsushita, Kanagawa (JP); Katsuhiko Ando, Tokyo (JP); Chitose Yoshida, Osaka (JP); Hirofumi Nakano, Tokyo (JP); Tsutomu Agatsuma, Tokyo (JP); Yutaka Kanda, Tokyo (JP)

Correspondence Address: Darby & Darby, PO Box 5257, New York, NY, 10150-5257, US

(21) Appl. No.: 10/483,439

(22) PCT Filed: Apr. 19, 2002
(86) PCT No.: PCT/JP02/03932

(30) Foreign Application Priority Data
Jul. 9, 2001 (JP) 2001-207187

(51) Int. Cl. C12N 9/99

(52) U.S. Cl. 435/184

(57) ABSTRACT

The present invention provides SH3 domain binding inhibitors comprising, as an active ingredient, a non-peptide compound exhibiting SH3 domain binding inhibitory activity, a low molecular weight compound with molecular weight less than 750 which exhibit SH3 domain binding inhibitory activity, in particular, a compound represented by the general formula (I) or (II) described above, a cytochalsin, etc., or pharmaceutically acceptable salts thereof. The present invention also provides compounds represented by the general formula (Va), (Vb) or (VI) described above or pharmaceutically acceptable salts thereof.

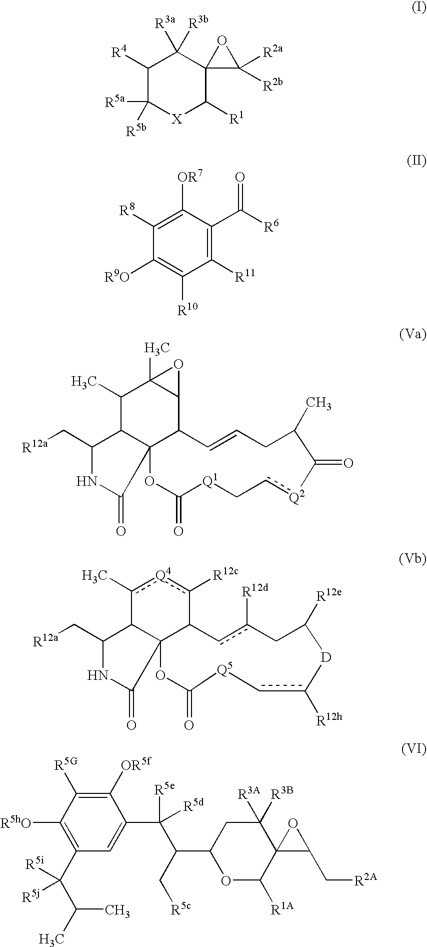

### TECHNICAL FIELD

The present invention relates to SH3 domain binding inhibitors that contain non-peptide compounds or their pharmaceutically acceptable salts as an active ingredient. The present invention also relates to useful compounds as SH3 domain binding inhibitors.

### BACKGROUND TECHNOLOGY

There are many receptors on the cell membrane that are activated by the extracellular stimulus and transmit the signals into the cell. Various signaling molecules are assembled on the cytosol side of the receptors and form a complex. Depending on the kind of the stimulus, each signaling molecule induces different physiological action. The structure of the domain plays an important role in the formation of a complex of signaling molecules. Among them, the Src homology domain 3 (SH3 domain) was discovered as a region with a high homology among Src family and it has been known that the domain consists of about 60 amino acids, and is present in various proteins and bound to a sequence containing proline (proline-rich sequence) by recognizing the sequence [Science, Vol. 252, p. 668 (1991), Science, Vol. 257, p. 803 (1992), FASEB J., Vol. 14, p. 231 (2000)]. HIV-1 Nef, p22-phox, p47-phox, Som68, Sos1, Zo1, Dynamin, c-Cb1, etc., have been known as proteins containing the proline-rich sequence(s) [e.g. EMBO J., Vol. 14, p. 484(1995), Proc. Natl. Acad. Sci. USA, Vol. 91, p. 10650 (1994), J. Immunol., Vol. 157, p. 1226 (1996), J. Biol. Chem., Vol.270, p. 9115 (1995), etc.] Proteins containing SH3 domains were found in all eukaryotes, viruses such as HIV, etc., and are considered to have universal functions. For example, the following proteins are known to contain SH3 domain(s): Fyn, which is one of Src family kinase; Ras-GAP, PLCγ, PI3K, Abl, Btk, Lyn, Hck, Fgr, Yes, etc., which are known to have enzyme activity; Grb2, Nck, Vav, etc., which are adaptor proteins without enzyme activity; p40-phox, p47-phox, p67-phox, etc., which are components of a NADH oxidase complex [Proc. Natl. Acad. Sci. USA, Vol. 91, p. 10650 (1994)]; and some others, that is, Txk, Tec, Tsk, Crk, Cortactin, etc. It has been speculated that the protein-protein interaction mediated by an SH3 domain has a role in the protein complex formation through an interaction due to a suitable affinity.

The protein-protein interactions mediated by an SH3 domain exist among the signaling molecules which have been considered as a cause of various diseases, such as cancer, AIDS (acquired immune deficiency syndrome), or allergy [Biopoly., Vol. 43, p. 383 (1997)]. These facts indicate that inhibitors of SH3 domain binding that inhibit the protein-protein interactions mediated by an SH3 domain (SH3 domain binding inhibitory activity) might be effective as a therapeutic agent for these diseases. The inhibitors may be used for various purposes, for example, for controlling a protein which cause a disease such as carcinogenesis in the body, in which SH3 domain binding is involved.

As to peptides, the following are reported: peptides with mutation in an SH3 domain binding sequence [Cell, Vol. 76, p. 933 (1994), Chem. Biol., Vol. 7, p. R3-R8 (2000)]; a method for screening peptides, which bind to an SH3 domain of various proteins containing SH3 domains such as Src, PI3K, or Ab1, from a combinatorial phage display library (WO95/24419, Japanese Translation of PCT Application [Tokuhyo] 2000-506522); peptides, which specifically bind to an SH3 domain of c-Crk, have been also reported (WO96/21011). Among peptide-like compounds that are obtained by modification of a peptide, the following compounds are known: spirolactam compounds which inhibit SH3 domain binding (WO98/54208) and compounds which are isolated from a biased combinatorial library in which a synthetic peptide containing an amino acid sequence having a proline skeleton is fixed [J. Am. Chem. Soc., Vol. 118, p. 287 (1996)]. Among those reported compounds, the smallest one have molecular weight of 790. However, K_{d} value thereof was 220 mmol/L and thus a binding ability thereof to the SH3 domain was week [J. Am. Chem. Soc., Vol. 118, p. 287 (1996)].

All the SH3 domain binding inhibitors reported so far are peptides or peptide-like compounds based on proline or other hydrophobic amino acids with 750 or more molecular weight. The peptides or the peptide-like compounds have been difficult to use as a therapeutic agent for a disease, in which SH3 domain binding is involved, because they are, for example, generally unstable in blood, poorly absorbed when orally administered, not easy to be taken into cells due to their large molecular weight, difficult to prepare and costly due to their structural complexity, etc.

UCS15A (Luminacin C2/SI-4228A) has been reported as the compound exhibiting inhibitory activity against bone resorption, bactercidal activity, immunosuppressive activity, anti-tricophytosis activity and anti-tumor activity [The Journal of Antibiotics, Vol. 53, p. 579 (2000), Japanese Published Unexamined Patent Application Nos. 116686/83, 22583/88, 293920/86, 294619/87, 48213/88 and 268888/96].

Also, cytochalasins (cytochalasin, Rosellichalasin, epoxycytochalasin, chaetoglobosin, penochalasin, aspochalasin, etc.) are known to possess biological activities such as an inhibitory activity against angiogenesis (WO98/41205).

### DISCLOSURE OF THE INVENTION

An object of the present invention is to provide SH3 domain binding inhibitors comprising a non-peptide compound or a pharmaceutically acceptable salt thereof as an active ingredient. Another object of the present invention is to provide compounds or pharmaceutically acceptable salts thereof which are useful as an SH3 domain binding inhibitor.

The present invention relates to the following [1]-[51].

[1] An SH3 domain binding inhibitor comprising a non-peptide compound exhibiting SH3 domain binding inhibitory activity or a pharmaceutically acceptable salt thereof as an active ingredient.

[2] The SH3 domain binding inhibitor according to [1], wherein said non-peptide compound is a low molecular weight compound with molecular weight of less than 750.

[3] The SH3 domain binding inhibitor according to [1] or [2], wherein said non-peptide compound exhibiting SH3 domain binding inhibitory activity is a compound represented by the general formula (I):

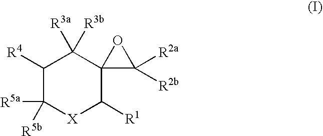

(wherein R¹, R^{3a}, R^{3b} and R⁴ may be the same or different and represent a hydrogen atom, hydroxy, carboxy, substituted or unsubstituted lower alkyl, substituted or unsubstituted lower alkoxy, or substituted or unsubstituted lower alkanoyloxy; or R^{3a} and R^{3b} are combined to represent an oxygen atom; R^{2a} and R^{2b} may be the same or different and represent a hydrogen atom, substituted or unsubstituted lower alkyl, or substituted or unsubstituted alkenyl; R^{5a} and R^{5b} may be the same or different and represent a hydrogen atom, hydroxy, carboxy, substituted or unsubstituted lower alkyl, substituted or unsubstituted lower alkoxy, substituted or unsubstituted lower alkenyl, substituted or unsubstituted lower alkanoyloxy, substituted or unsubstituted lower alkenoyloxy, or substituted or unsubstituted lower alkanoylaminocarbonyloxy; or R^{5a} and R^{5b} are combined to represent an oxygen atom; and X represents an oxygen atom or —CH₂—).

[4] The SH3 domain binding inhibitor according to [1] or [2], wherein said non-peptide compound exhibiting SH3 domain binding inhibitory activity is a compound represented by the general formula (II): 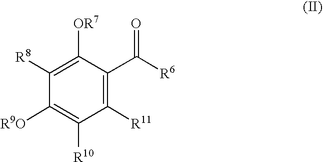
(wherein R⁶ represents a hydrogen atom, substituted or unsubstituted lower alkyl, or substituted or unsubstituted lower alkenyl; R⁷ and R⁹ may be the same or different and represent a hydrogen atom, formyl, substituted or unsubstituted lower alkyl, substituted or unsubstituted lower alkanoyl, or substituted or unsubstituted lower aralkyl; and R⁸, R¹⁰ and R¹¹ may be the same or different and represent a hydrogen atom, halogen, hydroxy, carboxy, substituted or unsubstituted lower alkyl, substituted or unsubstituted lower alkoxy, substituted or unsubstituted lower alkanoyl, formyl, cyano, nitro, amino, mono- or di-lower alkylamino, substituted or unsubstituted lower alkanoylamino, or substituted or unsubstituted alkoxycarbonyl).

[5] The SH3 domain binding inhibitor according to [1] or [2], wherein said non-peptide compound exhibiting SH3 domain binding inhibitory activity is a cytochalasin.

[6] The SH3 domain binding inhibitor according to [1] or [2], wherein said non-peptide compound exhibiting SH3 domain binding inhibitory activity is a compound represented by the general formula (IIIa): 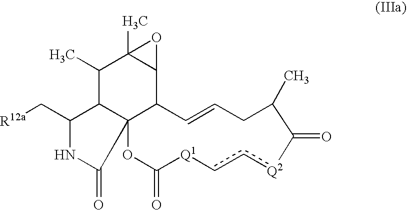
(wherein R^{12a} represents substituted or unsubstituted lower alkyl, substituted or unsubstituted aryl, or substituted or unsubstituted heteroaryl; Q¹ represents a single bond or an oxygen atom; 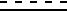
represents a single bond or a double bond; and when the 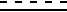
between Q² and a carbon atom adjacent thereto represents a double bond, =Q²— represents ═C(CH₃)—, and when the 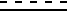
between Q² and a carbon atom adjacent thereto represents a single bond, -Q²- represents —C(OH)(CH₃)).

[7] The SH3 domain binding inhibitor according to [1] or [2], wherein said non-peptide compound exhibiting SH3 domain binding inhibitory activity is a compound represented by the general formula (IIIb):

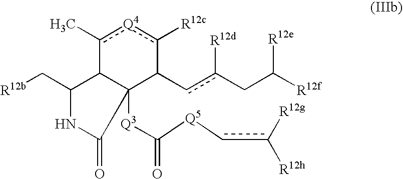

(wherein R^{12b} has the same meaning as the above-described R^{12a}; Q³ and Q⁵ may be the same or different and represent a single bond or an oxygen atom; 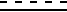
represents a single bond or a double bond; 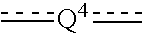 represents ═C(CH₃)—, —C(═CH₂)—, —CH(CH₃)— or —C(CH₃)═; R^{12c} and R^{12h} may be the same or different and represent a hydrogen atom or hydroxy; R^{12d} and R^{12e} may be the same or different and represent a hydrogen atom or methyl; and R^{12f} and R^{12g} represent formyl, or R^{12f} and R^{12g} are combined and 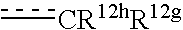
and CHR^{12e}R^{12f} form 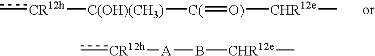
(wherein A and B may be the same or different and represent —CH(OH)—, —CH₂— or —C(═O)—)).

[8] The SH3 domain binding inhibitor according to [3], wherein X represents an oxygen atom; R^{2a}, R^{3a}, R⁴ and R^{5b} are hydrogen atoms; R¹ and R^{3b} may be the same or different and represent hydroxy, carboxy, substituted or unsubstituted lower alkoxy, or substituted or unsubstituted lower alkanoyloxy; R^{2b} represents substituted or unsubstituted lower alkyl; and R^{5a} represents the general formula (IV):

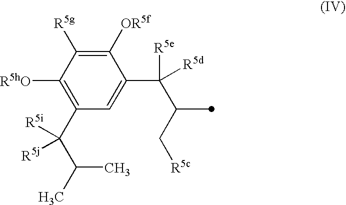

(wherein R^{5c} represents substituted or unsubstituted lower alkyl; R^{5d} and R^{5e} may be the same or different and represent a hydrogen atom, hydroxy, or substituted or unsubstituted lower alkoxy, or R^{5d} and R^{5e} are combined to represent an oxygen atom; R^{5f} and R^{5h} represent a hydrogen atom, formyl, substituted or unsubstituted lower alkyl, or substituted or unsubstituted lower alkanoyl; R^{5g} represents formyl, —CH═NQ (wherein Q represents substituted or unsubstituted lower alkoxy, substituted or unsubstituted aryloxy, substituted or unsubstituted aralkyloxy, substituted or unsubstituted lower alkylamino, substituted or unsubstituted arylamino, or substituted or unsubstituted arylsulfonylamino), substituted or unsubstituted lower alkyl, or substituted or unsubstituted lower alkanoyl; and R⁵ⁱ and R^{5j} may be the same or different and represent a hydrogen atom, hydroxy, or substituted or unsubstituted lower alkoxy, or R⁵ⁱ and R^{5j} are combined to represent an oxygen atom).

[9] A compound represented by the general formula (Va): 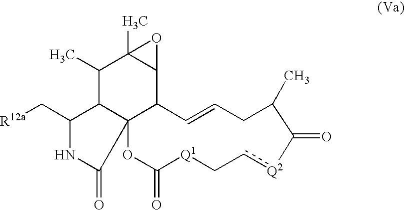
(wherein 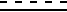
   represents a single bond or a double bond; and R^{12a}, Q¹ and Q² have the same meanings as defined above, respectively), or a pharmaceutically acceptable salt thereof.

[10] A compound represented by the general formula (Vb): 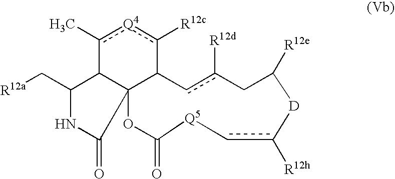
(wherein 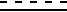
represents a single bond or a double bond; R^{12b}, R^{12c}, R^{12d}, R^{12e}, R^{12h}, Q⁵ and 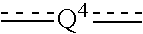
have the same meanings as defined above, respectively; and 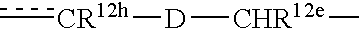
represents 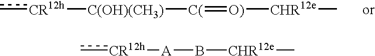
(wherein A and B have the same meanings as defined above, respectively)), or a pharmaceutically acceptable salt thereof.

[11] A compound represented by the general formula (VI) 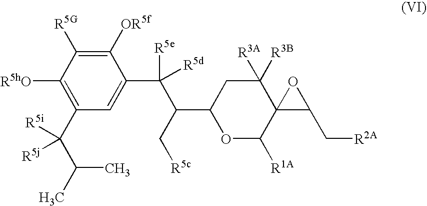
(wherein R^{1A}, R^{3A} and R^{3B} maybe the same or different and represent a hydrogen atom, hydroxy, carboxy, substituted or unsubstituted lower alkoxy, or substituted or unsubstituted lower alkanoyloxy, or R^{3A} and R^{3B} are combined to represent an oxygen atom; R^{2A} represents substituted or unsubstituted lower alkyl; R^{5c}, R^{5d}, R^{5e}, R^{5f}, R^{5h}, R⁵ⁱ and R^{5j} have the same meanings as defined above, respectively; and R^{5G} represents formyl, hydroxymethyl, substituted or unsubstituted lower alkoxymethyl, substituted or unsubstituted lower alkanoyloxymethyl, substituted or unsubstituted lower alkanoylmethyl, or —CH═NQ^{A} (wherein QA represents substituted or unsubstituted lower alkoxy, substituted or unsubstituted aryloxy, or substituted or unsubstituted aralkyloxy), with the proviso that when R^{5G} is formyl and one of R^{3A} and R^{3B} is a hydrogen atom, the other is not hydroxy], or a pharmaceutically acceptable salt thereof.

[12] A pharmaceutical composition comprising the compound according to [9] or [10] or a pharmaceutically acceptable salt thereof as an active ingredient.

[13] A pharmaceutical composition comprising the compound according to [11] or a pharmaceutically acceptable salt thereof as an active ingredient.

[14] An SH3 domain binding inhibitor comprising the compound according to [9] or [10] or a pharmaceutically acceptable salt thereof as an active ingredient.

[15] An SH3 domain binding inhibitor comprising the compound according to [11] or a pharmaceutically acceptable salt thereof as an active ingredient.

[16] The SH3 domain binding inhibitor according to any one of [1]-[8], [14] and [15], wherein said SH3 domain binding is an interaction between a protein containing an SH3 domain and a protein containing a proline-rich sequence.

[17] The SH3 domain binding inhibitor according to [16], wherein the protein containing an SH3 domain and/or the protein containing a proline-rich sequence are/is virus-derived protein(s).

[18] The SH3 domain binding inhibitor according to [17], wherein said virus-derived protein is a retrovirus-derived protein, a hepatitis virus-derived protein or a herpes virus-derived protein.

[19] The SH3 domain binding inhibitor according to [16], wherein said protein containing an SH3 domain is Src, Yes, Fgr, Hck, Lck, Abl, Fyn, Lyn, Blk, Yrk, Ras-GAP, PLCγ, PI3K, Tec, Txk/Rlk, Tsk/Emt/Itk, Btk, Crk, Grb2, Nck, Vav, STAT, Cortactin, p40-phox, p67-phox, p47-phox, a TCR signaling molecule (TCRsm), or a β chain or a γ chain of IL-2R.

[20] The SH3 domain binding inhibitor according to [16] or [19], wherein said protein containing a proline-rich sequence is HIV-1Nef, p22-phox, p47-phox, Sam68, Sos1, Dynamin, c-Cb1, ZO1, pX ORF I, LHDAg, NS5A, pORF3, ICP10, LMP2A, Tip or Tio.

[21] The SH3 domain binding inhibitor according to [16], wherein said interaction between a protein containing an SH3 domain and a protein containing a proline-rich sequence is an interaction between Grb2 and Sos1, Fyn and Sam68, Src and Sam68, PLCγ and Sam68, Grb2 and Sam68, Lyn and HIV-1Nef, Hck and HIV-1Nef, TCRsm and HIV-1Nef, p47-phox and p22-phox, p67-phox and p47-phox, Lyn and Dynamin, Cortactin and ZO1, Lyn and c-Cbl, a β chain or a γ chain of IL-2R and pX ORF I, Grb2 and NS5A, Src and pORF3, Hck and pORF3, Fyn and pORF3, PI3K and pORF3, PLCγ and pORF3, Grb2 and pORF3, Grb2 and ICP10, Lyn and LMP2A, Lck and Tip, Lyn and Tio, Hck and Tio, Lck and Tio, Src and Tio, Fyn and Tio, or Yes and Tio.

[22] The SH3 domain binding inhibitor according to [16], wherein said interaction between a protein containing an SH3 domain and a protein containing a proline-rich sequence is an interaction between Grb2 and Sos1, Fyn and Sam68, Src and Sam68, PLCγ and Sam68, Grb2 and Sam68, Lyn and HIV-1Nef, or Cortactin and ZO1.

[23] A microorganism for producing the compound according to [9] or [10], selected from the group consisting of Xylariales filamentous fungus MPC1005 (Accession No.: FERM BP-7980), Aspergillus sp. MPC1006 (Accession No.: FERM BP-7899) and Aspergillus sp. MPC1009 (Accession No.: FERM BP-7900).

[24] An anti-tumor agent comprising the compound according to [9] or [10] or a pharmaceutically acceptable salt thereof as an active ingredient.

[25] An anti-tumor agent comprising the compound according to [11] or a pharmaceutically acceptable salt thereof as an active ingredient.

[26] A therapeutic agent for an allergic disease comprising the compound according to [9] or [10] or a pharmaceutically acceptable salt thereof as an active ingredient.

[27] A therapeutic agent for an allergic disease comprising the compound according to [11] or a pharmaceutically acceptable salt thereof as an active ingredient.

[28] A therapeutic agent for a viral disease comprising the compound according to claim [9] or [10] or a pharmaceutically acceptable salt thereof as an active ingredient.

[29] A therapeutic agent for a viral disease comprising the compound according to [11] or a pharmaceutically acceptable salt thereof as an active ingredient.

[30] A therapeutic agent for AIDS comprising the compound according to [9] or [10] or a pharmaceutically acceptable salt thereof as an active ingredient.

[31] A therapeutic agent for AIDS comprising the compound according to [11] or a pharmaceutically acceptable salt thereof as an active ingredient.

[32] Use of the compound according to [9] or [10] or a pharmaceutically acceptable salt thereof for the manufacture of an SH3 domain binding inhibitor.

[33] Use of the compound according to [11] or a pharmaceutically acceptable salt thereof for the manufacture of an SH3 domain binding inhibitor.

[34] Use of the compound according to [9] or [10] or a pharmaceutically acceptable salt thereof for the manufacture of an anti-tumor agent.

[35] Use of the compound according to [11] or a pharmaceutically acceptable salt thereof for the manufacture of an anti-tumor agent.

[36] Use of the compound according to [9] or [10] or a pharmaceutically acceptable salt thereof for the manufacture of a therapeutic agent for an allergic disease.

[37] Use of the compound according to [11] or a pharmaceutically acceptable salt thereof for the manufacture of a therapeutic agent for an allergic disease.

[38] Use of the compound according to [9] or [10] or a pharmaceutically acceptable salt thereof for the manufacture of a therapeutic agent for a viral disease.

[39] Use of the compound according to [11] or a pharmaceutically acceptable salt thereof for the manufacture of a therapeutic agent for a viral disease.

[40] Use of the compound according to [9] or [10] or a pharmaceutically acceptable salt thereof for the manufacture of a therapeutic agent for AIDS.

[41] Use of the compound according to [11] or a pharmaceutically acceptable salt thereof for the manufacture of a therapeutic agent for AIDS.

[42] A method for treating and/or preventing a disease in which an SH3 domain binding is involved, which comprises administering an effective amount of the compound according to [9] or [10] or a pharmaceutically acceptable salt thereof.

[43] A method for treating and/or preventing a disease in which an SH3 domain binding is involved, which comprises administering an effective amount of the compound according to [11] or a pharmaceutically acceptable salt thereof.

[44] A method for treating a malignant tumor, which comprises administering an effective amount of the compound according to [9] or [10] or a pharmaceutically acceptable salt thereof.

[45] A method for treating a malignant tumor, which comprises administering an effective amount of the compound according to [11] or a pharmaceutically acceptable salt thereof.

[46] A method for treating and/or preventing an allergic disease, which comprises administering an effective amount of the compound according to [9] or [10] or a pharmaceutically acceptable salt thereof.

[47] A method for treating and/or preventing an allergic disease, which comprises administering an effective amount of the compound according to [11] or a pharmaceutically acceptable salt thereof.

[48] A method for treating a viral disease, which comprises administering an effective amount of the compound according to [9] or [10] or a pharmaceutically acceptable salt thereof.

[49] A method for treating a viral disease, which comprises administering an effective amount of the compound according to [11] or a pharmaceutically acceptable salt thereof.

[50] A method for treating AIDS, which comprises administering an effective amount of the compound according to [9] or [10] or a pharmaceutically acceptable salt thereof.

[51] A method for treating AIDS, which comprises administering an effective amount of the compound according to [11] or a pharmaceutically acceptable salt thereof.

[52] A method for treating AIDS, which comprises administering an effective amount of a compound exhibiting an SH3 domain binding inhibitory activity or a pharmaceutically acceptable salt thereof.

[53] A method for treating a viral disease, which comprises administering an effective amount of a compound exhibiting an SH3 domain binding inhibitory activity or a pharmaceutically acceptable salt thereof.

[54] A therapeutic agent for AIDS comprising a compound exhibiting an SH3 domain binding inhibitory activity or a pharmaceutically acceptable salt thereof as an active ingredient.

[55] A therapeutic agent for a viral diseases comprising a compound exhibiting an SH3 domain binding inhibitory activity or a pharmaceutically acceptable salt thereof as an active ingredient.

[56] Use of a compound exhibiting an SH3 domain binding inhibitory activity or a pharmaceutically acceptable salt thereof for the manufacture of a therapeutic agent for AIDS.

[57] Use of a compound exhibiting an SH3 domain binding inhibitory activity or a pharmaceutically acceptable salt thereof for the manufacture of a therapeutic agent for a viral disease.

Hereinafter, the compounds represented by general formulae (I), (II), (IIIa), (IIIb), (Va), (Vb) and (VI) are referred to as Compounds (I), (II), (IIIa), (IIIb), (Va), (Vb) and (VI), respectively.

In the definition of each group in general formulae (I), (II), (IIIa), (IIIb), (IV), (Va), (Vb) and (VI):
(1) The lower alkyl, and the lower alkyl moieties of the lower alkoxy, the lower alkoxycarbonyl, the lower alkoxymethyl, the lower alkylamino and the mono- or di-lower alkylamino include, for example, linear or branched alkyl having 1 to 8 carbon atoms, specifically methyl, ethyl, propyl, isopropyl, butyl, sec-butyl, tert-butyl, pentyl, isopentyl, neopentyl, hexyl, heptyl, octyl, etc.; and also include, for example, cyclic alkyl having 3 to 8 carbon atoms, specifically cyclopropyl, cyclobutyl, cyclopentyl, cyclohexyl, cycloheptyl, cyclooctyl, etc.

(2) The lower alkenyl includes, for example, linear or branched alkenyl having 2 to 8 carbon atoms, specifically vinyl, allyl, 1-propenyl, methacryl, crotyl, 1-butenyl, 3-butenyl, 2-pentenyl, 4-pentenyl, 2-hexenyl, 5-hexenyl, 1,5-dimethyl-4-hexenyl, etc.; and also includes alkenyl having 2 to 4 double bonds, specifically 1,3-butadienyl, 1,3-pentadienyl, 2,4-pentadienyl, 1,5-dimethyl-1,4-hexadienyl, 1,3,5-hexatrienyl, etc.

(3) The lower alkanoyl, and the lower alkanoyl moieties of the lower alkanoyloxy, the lower alkanoyloxymethyl, the lower alkanoylmethyl, the lower alkanoylamino and the lower alkanoylaminocarbonyloxy include, for example, linear or branched alkanoyl having 2 to 8 carbon atoms, specifically acetyl, propionyl, butyryl, isobutyryl, valeryl, isovaleryl, pivaloyl, hexanoyl, heptanoyl, octanoyl, etc.

(4) The aryl, and the aryl moieties of the arylamino, the arylsulfonylamino and the aryloxy include, for example, aryl having 6 to 14 carbon atoms, specifically phenyl, naphthyl, anthryl, etc.

(5) The aralkyl and the aralkyl moiety of the aralkyloxy include, for example, aralkyl having 7 to 15 carbon atoms, specifically benzyl, phenethyl, benzhydryl, naphthylmethyl, etc.

(6) The heteroaryl includes, for example, 5- or 6-membered monocyclic aromatic heterocyclic groups containing at least one heteroatom selected from a nitrogen atom, an oxygen atom and a sulfur atom, and bicyclic or tricyclic condensed aromatic heterocyclic groups containing at least one heteroatom selected from a nitrogen atom, an oxygen atom and a sulfur atom, in which 3- to 8-membered rings are condensed, more specifically, for example, pyridyl, pyradinyl, pyrimidinyl, pyridazinyl, quinolinyl, isoquinolinyl, phthalazinyl, quinazolinyl, quinoxalinyl, naphthyrizinyl, cinnolinyl, pyrrolyl, pyrazolyl, imidazolyl, triazolyl, tetrazolyl, thienyl, furyl, thiazolyl, oxazolyl, indolyl, indazolyl, benzimidazolyl, benzotriazolyl, benzothiazolyl, benzoxazolyl, purinyl, etc.

(7) The halogen means fluorine, chlorine, bromine and iodine atoms.

(8) The lower alkenyl moiety of the lower alkenoyloxyl has the same meaning as the above-described lower alkenyl (2).

(9) The substituents in the substituted aryl, the substituted aryloxy, the substituted arylamino, the substituted arylsulfonylamino, the substituted aralkyl and the substituted aralkyloxy, can be 1 to 5 substituents which are the same or different, and are, for example, hydroxy, halogen, formyl, substituted or unsubstituted lower alkyl, substituted or unsubstituted lower alkoxy, lower alkanoyl, etc. The positions of the substituents are not limited. The halogen, the lower alkyl and the lower alkyl moiety of the lower alkoxy, and the lower alkanoyl mentioned herein, have the same meanings as the above-described halogen (7), the lower alkyl (1) and the lower alkanoyl (3), respectively. The substituents in the substituted lower alkyl and the substituted alkoxy mentioned herein, can be 1 to 3 substituents, and are, for example, hydroxy, lower alkoxy [the lower alkyl moiety of the lower alkoxy has the same meaning as the above-described lower alkyl (1)], halogen [the halogen has the same meaning as the above-described halogen (7)], etc.

(10) The substituents in the substituted lower alkyl, the substituted lower alkoxy, the substituted lower alkoxycarbonyl, the substituted lower alkoxymethyl, the substituted lower alkanoyl, the substituted lower alkanoyloxy, the substituted lower alkanoyloxymethyl, the substituted lower alkanoylmethyl, the substituted lower alkenoyloxy, the substituted lower alkanoylamino, the substituted lower alkanoylaminocarbonyloxy, the substituted lower alkylamino and the substituted lower alkenyl, can be 1 to 3 substituents which are the same or different, and are, for example, hydroxy, lower alkyl, lower alkoxy, carboxy, oxo, amino, epoxy, substituted or unsubstituted lower alkanoyl, substituted or unsubstituted lower alkanoyloxy, substituted or unsubstituted aryl, substituted or unsubstituted aroyl, etc. The positions of the substituents are not limited. The lower alkanoyl and the lower alkanoyl moiety of the lower alkanoyloxy, the lower alkyl and the lower alkyl moiety of the lower alkoxy, and the aryl and the aryl moiety of the aroyl mentioned herein, have the same meanings as the above-described lower alkanoyl (3), the lower alkyl (1), and the aryl (4), respectively. The substituents in the substituted aryl and the substituted aroyl have the same meanings as the above-described substituents (9) of the substituted aryl. The substituents in the substituted lower alkanoyl and the substituted lower alkanoyloxy mentioned herein, can be 1 to 3 substituents, and are, for example, hydroxy, lower alkoxy [the lower alkyl moiety of the lower alkoxy has the same meaning as the above-described lower alkyl (1)], and halogen [the halogen has the same meaning as the above-described halogen (7)], etc.

(11) The cytochalasins include, for example, cytochalasin, Rosellichalasin, epoxycytochalasin, chaetoglobosin, penochalasin, aspochalasin, etc.

(12) The SH3 domain binding inhibition means inhibition of a protein-protein interaction mediated by an SH3 domain.

(13) The SH3 domain binding includes, for example, any interactions between a protein containing an SH3 domain, and a protein containing a proline-rich sequence or the like, and more specifically, for example, the interactions wherein the protein containing an SH3 domain and/or the protein containing a proline-rich sequence are/is virus-derived protein(s).

The virus-derived proteins include, for example, proteins derived from a retrovirus such as human immunodeficiency virus type 1 (HIV-1) or human T-lymphotropic virus type 1 (HTLV-1), proteins derived from a hepatitis virus such as hepatitis delta virus (HDV), hepatitis C virus (HCV) or hepatitis E virus (NEV), and proteins derived from a herpes viruse such as herpes simplex virus type 2 (HSV-2), EB virus (EBV), herpesvirus saimiri (H. saimiri) or herpesvirus ateles (H. ateles), etc.

The proteins containing an SH3 domain include, for example, the Src family proteins of non-receptor type tyrosine kinase, such as Src, Yes, Fgr, Hck, Lck, Abl, Fyn (fgr/yes-related novel gene), Lyn (lgr/yes-related novel gene), Blk (B-cell lymphocyte kinase) and Yrk (Yes-related kinase); proteins having enzymatic activity, such as Ras-GAP (ras-GTPase-activating protein), PLCγ (phospholipase C-gamma) and PI3K (phosphatidylinositol 3-kinase); Tec family proteins of non-receptor type tyrosine kinase, such as Tec, Txk/Rlk, Tsk/Emt/Itk and Btk (Bruton's tyrosine kinase); adaptor proteins, such as Crk (CT-10bregulated), Grb2 (growth factor receptor-bound protein 2), Nck and Vav; transcription factors, such as STAT (signal transducers and activators of transcription); proteins involved in actin skeleton formation, such as Cortactin; components of a NADPH oxidase complex, such as p40-phox, p67-phox and p47-phox; T-cell receptor signaling molecules, such as TCRsm [Curr. Biol., Vol. 11, p. 1294 (2001)]; a β chain or a γ chain of IL-2R (interleukin-2 receptor) [J. Virol., Vol. 74, p. 9828 (2000)]; etc.

The proteins containing a proline-rich sequence include, for example, Nef proteins which are involved in viral infection and/or growth, replication of HIV, or the like, such as HIV-1Nef; components of a NADPH oxidase complex, such as p22-phox and p47-phox; proteins involved in the cell cycles, such as Sam68 (src-associated mitotic substrate 68); adaptor proteins, such as Sos1 (son of sevenless), Dynamin and c-Cbl (casitas B-lineage lymphoma); proteins involved in the cytoskeleton, such as ZO1; proteins involved in duration of virus infection or the like, such as pX ORF-I (open reading frame I of the pX regione) of HTLV-1 [J. Virol., Vol. 74, p. 9828 (2000)]; proteins involved in viral RNA synthesis, such as LHDAg (large hepatitis delta antigen) [J. Virol., Vol.72, p. 2089 (1998)], NS5A [Proc. Natl. Acad. Sci. USA, Vol. 961, p. 5533 (1999)], pORF3 [J. Biol. Chem., Vol. 276, p. 42389 (2002)] and ICP10 [J. Biol. Chem., Vol. 271, p. 17021 (1996)); proteins involved in the latency and/or penetration of virus in host cells, such as LMP2A [Exp. Cell Res., Vol.257, p. 332 (2000), J. Virol., Vol.69, p. 7814 (1995)], Tip [J. Virol., Vol. 72, p. 2607 (1998)], and Tio [J. Virol., Vol. 73, p. 4631 (1999)]; etc.

The interactions between a protein containing an SH3 domain and a protein containing an proline-rich sequence include, for example, interactions between Fyn and Sam68 [J. Biol. Chem., Vol. 272, p. 6214 (1997)], Src and Sam68 [Mol. Cell Biol., Vol. 15, p. 186 (1995)], Grb2 and Sos1 [Nature, Vol. 363, p. 83 (1993)], PLCγ and Sam68 [Oncogene, Vol. 18, p. 4647 (1999)], Grb2 and Sam68 [Mol. CellBiol., Vol. 15, p. 186 (1995)], Lyn and HIV-1Nef, or Hck and HIV-1Nef [EMBO. J., Vol. 14, p. 484 (1995), Virology, Vol. 262, p. 55 (1999)], TCRsm and HIV-1Nef [Curr. Biol., Vol. 11, p. 1294 (2001)], p47-phox and p22-phox [Proc. Natl. Acad. Sci. USA, Vol. 91, p. 10650 (1994)], p67-phox and p47-phox [Proc. Natl. Acad. Sci. USA, Vol. 91, p. 10650 (1994)], Lyn and Dynamin [J. Immunol., Vol. 157, p. 1226 (1996)], Lyn and c-Cbl [J. Biol. Chem., Vol. 270, p. 9115 (1995)], Cortactin and Zo1 [J. Biol. Chem., Vol. 273, p. 29672 (1998)], a P chain or a y chain of IL-2R and pX ORF I [J. Virol., Vol. 74, p. 9828 (2000)], Grb2 and NS5A [Proc. Natl. Acad. Sci. USA, Vol. 96, p. 5533 (1999)], Src and pORF3, Hck and pORF3, Fyn and pORF3, PI3K and pORF3, PLCa and pORF3, or Grb2 and pORF3 [J. Biol. Chem., Vol. 276, p. 42389 (2002)], Grb2 and ICP10 [J. Biol. Chem., Vol. 271, p. 17021 (1996)], Lyn and LMP2A [Exp. Cell Res., Vol. 257, p. 332 (2000), J. Virol., Vol. 69, p. 7814 (1995)], Lck and Tip [J. Virol., Vol. 72, p. 2607 (1998)], Lyn and Tio, Hck and Tio, Lck and Tio, Src and Tio, Fyn and Tio, or Yes and Tio [J. Virol., Vol. 73, p. 4631 (1999)], etc.

The pharmaceutically acceptable salts for non-peptide compounds include, for example, pharmaceutically acceptable acid addition salts, metal salts, ammonium salts, organic amine addition salts, amino acids addition salts, etc.

The pharmaceutically acceptable acid addition salts can be inorganic acid addition salts such as hydrochloride, sulfate, nitrate and phosphate; organic acid addition salts such as acetate, maleate, fumarate and citrate; etc. The pharmaceutically acceptable metal salts can be alkali metal salts such as sodium salt and potassium salt; alkaline earth metal salts such as magnesium salt and calcium salt; aluminum salt; zinc salt; etc. The pharmaceutically acceptable ammonium salts can be salts of ammonium; tetramethylammonium; etc. The pharmaceutically acceptable organic amine addition salts can be salts with morpholine, piperidine etc. The pharmaceutically acceptable amino acid addition salts can be addition salts of glycine; phenylalanine; lysine; aspartic acid; glutamic acid; etc.

Here we explain the production methods for the non-peptide compounds used in the present invention.

In the production methods described below, if the defined groups undergo changes under the reaction conditions or are not suitable to carry out the methods, production can be easily performed by applying means usually used in synthetic organic chemistry, such as protection and de-protection of the functional groups [for example, Protective Groups in Organic Synthesis, third edition, by T. W. Green, John Wiley & Sons Inc., (1999)]. And also, if necessary, it is possible to change the order of the reaction steps such as the introduction of the substituents.

The non-peptide compounds used in the present invention can be produced, for example, by a series of the reactions described below.

Production Method 1:

Among the non-peptide compounds used in the present invention, Compound (VI) can beproducedbythe following series of reaction steps.

Step 1:

Among Compounds (VI), Compound (VIa) in which R^{5G} is hydroxymethyl can be produced from Compound (VII) by the following reaction steps. 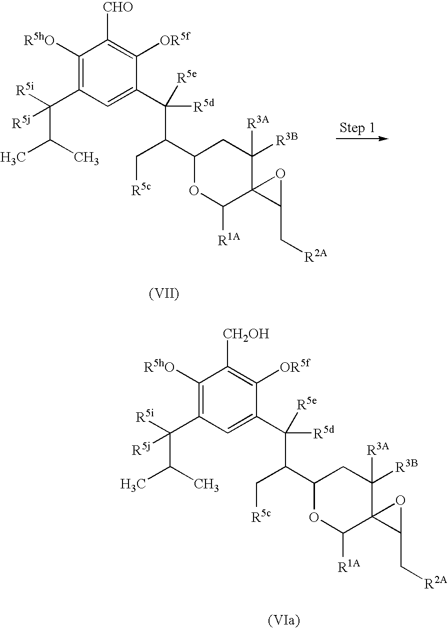
(wherein R^{1A}, R^{2A}, R^{3A}, R^{3B}, R^{5c}, R^{5d}, R^{5e}, R^{5f}, R^{5h}, R⁵ⁱ and R^{5j} have the same meanings as defined above, respectively)

Compound (VIa) can be obtained by treating Compound (VII) with a reducing agent in inert solvent.

The reducing agent can be, for example, sodium borohydride, lithium aluminum hydride, diisobutyl aluminum hydride, sodium bis(2-methoxyethoxy)aluminum hydride, etc., and is used in an amount of 1-10 equivalents based on Compound (VII).

The inert solvent can be, for example, methanol, ethanol, chloroform, dichloromethane, tetrahydrofuran (THF), diethyl ether, toluene, dimethylformamide (DMF), etc.

The reaction can be carried out at a temperature between 0° C. and the boiling point of the solvent used, usually for 1 minute to 24 hours.

Step 2:

Among Compounds (VI), Compound (VIb), in which R^{5G} is substituted or unsubstituted lower alkoxymethyl, or substituted or unsubstituted lower alkanoyloxymethyl, can be produced from Compound (VIa) obtained by Step 1 by the following reaction steps. 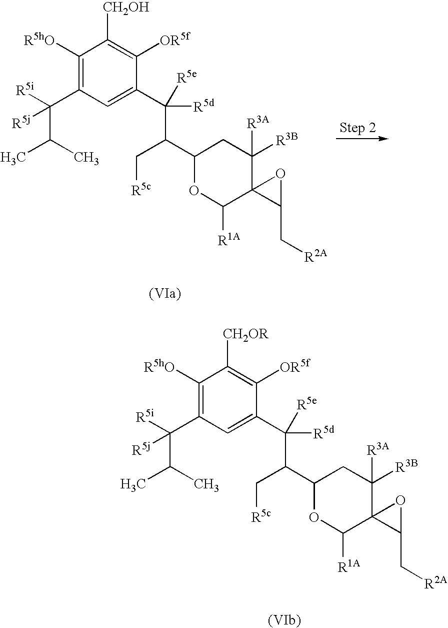
(wherein R^{1A}, R^{2A}, R^{3A}, R^{3B}, R^{5c}, R^{5d}, R^{5e}, R^{5f}, R^{5h}, R⁵ⁱ and R^{5j} have the same meanings as defined above, respectively; R represents substituted or unsubstituted lower alkyl [the lower alkyl has the same meaning as above-described the lower alkyl(1) and the substituents in the substituted lower alkyl have the same meanings as above-described the substituents (10) of the substituted lower alkyl] and substituted or unsubstituted lower alkanoyl [the lower alkanoyl has the same meaning as above-described the lower alkanoyl (3) and the substituents have the same meanings as above-described the substituents (10) of the substituted lower alkanoyl]}

Compound (VIb) can be obtained from the Compound (VIa) which is obtained in Step 1 by reacting with 1-20 equivalents of R-Z (wherein R has the same meanings as defined above, and Z represents a chlorine atom, a bromine atom or an iodine atom) in an inert solvent in the presence of a base. And also, when R is substituted or unsubstituted lower alkanoyl, R₂O (wherein R has the same meanings as defined above), which is carboxylic acid unhydrate, can be used instead of R-Z.

The base can be, for example, pyridine, triethylamine, potassium carbonate, cesium carbonate, sodium hydride, etc., and is used in an amount of 1-50 equivalents based on Compound (VIa) or as the solvent.

The inert solvent can be, for example, chloroform, dichloromethane, acetone, diethyl ether, acetonitrile, THF, DMF, etc.

The reaction can be carried out at a temperature between 0° C. and the boiling point of the solvent used and usually for 5 minutes to 24 hours.

Step 3:

Among Compounds (VI), Compound (VIc), in which R^{5G} is —CH═NQ^{A} (wherein Q^{A} has the same meaning as defined above), can be obtained from Compound (VII) by the following reaction steps. 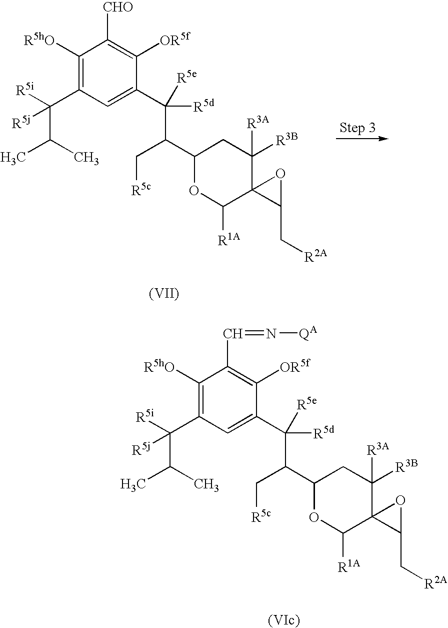
(wherein R^{1A}, R^{2A}, R^{3A}, R^{3B}, R^{5c}, R^{5d}, R^{5e}, R^{5f}, R^{5h}, R⁵ⁱ, R^{5j} and Q^{A} have the same meanings as defined above, respectively).

Compound (VIc) can be obtained from Compound (VII) by reacting with 1-10 equivalents of Q^{A}NH₂ (wherein Q^{A} has the same meaning as defined above) in an inert solvent, if necessary, in the presence of a base.

The base can be, for example, pyridine, triethylamine, etc., and is used in an amount of 1-20 equivalents based on Compound (VII).

The inert solvent can be, for example, chloroform, dichloromethane, acetone, diethyl ether, acetonitrile, THF, DMF, etc.

The reaction can be carried out at a temperature between 0° C. and the boiling point of the solvent used and usually for 5 minutes to 24 hours.

Compound (VII) which is the raw material in Production method 1, for example, can be obtained according to the method as described in The Journal of Antibiotics, Vol.53, p. 579 (2000) or Japanese Published Unexamined Patent Application No. 116686/83 or methods similar to those.

Production Method 2:

Compound (II) can be produced from Compound (VIII) by the following method: 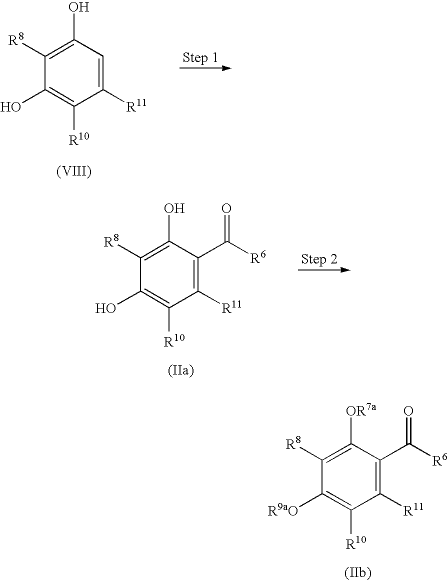
(wherein R⁶, R⁸, R¹⁰ and R¹¹ have the same meanings as defined above, respectively; R^{7a} and R^{9a} have the same meanings as above-defined R⁷ and R⁹, respectively, with the proviso that R⁷ and R⁹ are not an hydrogen atom at the same time)
Step 1:

Compound (IIa) can be obtained from the Compound (VIII) by reacting with 1-10 equivalents of R⁶CO₂H (wherein R⁶ has the same meaning as defined above) or derivatives thereof in an inert solvent in the presence of an acid.

The acid can be, for example, an organic acid such as formic acid, acetic acid and trifluoroacetic acid; an inorganic acid such as hydrochloric acid, sulfuric acid, nitric acid and phosphoric acid; and Lewis acid such as titanium tetrachloride and boron trifluoride diethyl etherate complex; etc. Among them, boron trifluoride diethyl etherate complex is preferred. The acid is used in an amount of 1-100 equivalents based on the Compound (VIII) or also as a solvent.

The inert solvent can be, for example, chloroform, dichloromethane, THF, DMF, etc.

The reaction can be carried out at the temperature between −30° C. and the boiling point of the solvent used and usually for 1 minute to 24 hours.

Compound (VIII) which is the raw material is commercially available, or can be obtained according to the method described in J. Am. Chem. Soc., Vol. 122, p. 3071 (2000) or a method similar to that.

Step 2:

Compound (IIb) can be obtained from Compound (IIa), which is obtained in Step 1 of Production method 2, by reacting with 1-20 equivalents of R^{9a}Z (R^{9a} and Z have the same meanings as defined above, respectively) in an inert solvent in the presence of a base.

The base can be, for example, pyridine, triethylamine, potassium carbonate, cesium carbonate, calcium carbonate, sodium hydroxide, etc. The base is used in an amount of 1-5 equivalents based on Compound (IIa).

The inert solvent can be, for example, chloroform, dichloromethane, acetone, diethylether, acetonitrile, THF, DMF etc.

The reaction can be carried out at the temperature between 0° C. and the boiling point of the solvent used and usually for 5 minute to 24 hours.

Production Method 3:

Compound (IIIa) and Compound (IIIb) can be obtained, for example, according to the methods described in Japanese Published Unexamined Patent Application No. 114776/98, WO98/41205, Helv. Chem. Acta, Vol. 62, p. 1501 (1979), etc., or methods similar to those.

Production Method 4:

Compound (I) can be obtained, for example, according to the method above-described in Production method 1, J. Am. Chem. Soc., Vol. 107, p. 256 (1985), ibid., Vol. 94, p. 2549 (1972), The Journal of Antibiotics, Vol.53, p. 579 (2000), Japanese Published Unexamined Patent Application No. 116686/83, etc., or methods similar to those.

Among the non-peptide compounds used in the present invention, Compound (Va) and Compound (Vb) (cytochalasin derivatives) can be produced by the following culturing methods.

Production Method 5:

The cytochalasin derivatives are produced by culturing the microorganisms capable of producing the cytocharasin derivatives in the culture and recovering them from the culture in which the cytochalasin derivatives are produced and accumulated.

The microorganisms capable of producing the cytochalasin derivatives can be used any microorganic strains capable of producing the cytocharasin derivatives for the production. Even if these microorganic strains are spontaneously or artificially mutated, for example, by UV-irradiation, X-ray irradiation, or mutagen treatment, they can be used for the present invention as long as they can produce the cytochasin derivatives.

More specifically, Aspergillus sp. MPC1006, Aspergillus sp. MPC1009, Xylariales filamentous fungus MPC1005, etc., can be used for the present invention.

General culture methods for fungi can be applied to culture the microorganisms capable of producing the cytochalasin derivatives in the present invention. The culture medium can be synthetic or natural as long as they contain proper amounts of such as carbon sources, nitrogen sources and inorganic substances for assimilation of microorganisms.

The carbon sources can be glucose, starch, dextrin, mannose, fructose, sucrose, lactose, xylose, arabinose, mannitol, molasses, etc. These can be used singly or in combinations. Furthermore, depending on the ability of assimilation of the microorganic strains, hydrocarbons, alcohols, organic acids, etc. can be used.

The nitrogen sources can be ammonium chloride, ammonium nitrate, ammonium sulfate, sodium nitrate, urea, peptone, meat extracts, yeast extracts, dried yeast, Corn Steep Liquor, soy bean flour, casamino acid. These can be used singly or in combinations.

If necessary, inorganic salts, such as sodium chloride, potassium chloride, magnesium sulfate, calcium carbonate, potassium dihydrogen phosphate, magnesium phosphate.8H₂O, ferrous sulfate, calcium chloride, manganese sulfate, zinc sulfate, cupper sulfate, etc., can be added thereto. Furthermore, trace amounts of components can be added appropriately thereto, to promote the growth of the microorganic strains or the production of the cytochalasin derivatives.

The liquid culture, especially the deep stirring culture method, is suitable for the growth of the microorganisms used in the present invention. The temperature for the incubation can be 16-37° C., preferably 25-32° C., and the pH of culture medium can be pH 4-10, preferably pH 6-8. Adjustment of the pH of the culture medium is carried out by using ammonia water or ammonium carbonate solutions. The incubation is usually terminated after 1-10 days but it is preferred to terminate when the cytochalasin derivatives are produced and accumulated in the media and in the microbial cells, and reach to the maximum concentration in the culture.

The isolation and purification of the cytochalasin derivatives accumulated in the culture are carried out by the general methods for the isolation and purification of the metabolites of microorganisms from the cultures. For example, the culture is filtered to separate microbial cells and the culture supernatant, and the cells are extracted with a solvent such as chloroform, acetone, methanol, etc. Then, the extracts and the culture supernatant are combined and pass through a column of polystyrene absorbent such as, for example, Diaion HP20 (Mitsubishi Chemical Co.), to absorb the active ingredients. And then, the column is eluted with methanol, acetone, etc., and the eluent is concentrated. The cytochalasin derivatives are obtained from the resulting concentrate by treating with column chromatography using octadecyl group bound silica gel (ODS), high speed liquid chromatography, column chromatography using silica gel, etc. Detection of the cytochalasin derivatives in the culture and during the isolation and purification processes can be carried out by thin layer chromatography followed by the treatment with the iodine reagent.

The inventers of the present invention discovered that MPC1006 and MPC1009, which have been newly isolated from soil and belong to Aspergillus sp., produced the cytochalasin derivatives exhibiting SH3 binding inhibitory activity.

The strain MPC1006, which represents the strains that produce the compounds of the present invention and was isolated from soil, has following characteristics:

1. Macroscopic Observations

When cultured at 25° C. on a malt extract agar plate, the colony diameter is 38-39 mm at day 7 and 65-68 mm at day 11. At day 11, the color of the center surface of the colonies is very light reddish yellow and the outside surface is very light yellow. The color of the back of the colonies is reddish yellow at the center and very light yellow outside.

When cultured at 25° C. on a potato-glucose-agar plate, the colony diameter is 38-40 mm at day 7 and 46-48 mm at day 11. At day 11, the color of the center surface of the colonies is blight grey-yellow and the outside surface is very light yellow with occasional white spots. The color of the back of the colonies is dark yellow at the center and light yellow outside. This strain grows in the range of 11.5-34° C., with the optimum growth at around 28.5° C. and in the rage of the pH 3.5-11.5 with the optimum growth at around pH 7.5.

2. Microscopic Observations

When cultured at 25° C. for 7 days on a malt extract agar plate, the light-microscopic observations of this strain are as follows:

The hypha, that contains septum, is 1.0-3.0 μm wide, smooth, colorless and well branched. The conidiophore, that does not contain septum, is 3.5-5.0 μm wide and 420-700 μm long, smooth, colorless and no branch. The tip of the codiophore is round forming a vesicle with diameter 12.5-25.0 μm and covered with metulae. The length of the metulae is 5.5-8.0 mm and on the tips of the metulae, bowling-pin shaped phialides (3.5-6.0 μm length) are formed. From the phialides, the conidia are formed in chains in endoconidium (phialo-type). The phialo-type conidium is a single cell, light yellow green color, globular or semi-globular shaped with smooth surface and with a diameter 1.5-3.00 μm. In this strain only anamorph as described above is observed but not the teleomorph.

The other strain, MPC1009, which also represents the strains that produce the compound of the present invention and was isolated from soil, has following characteristics:

1. Macroscopic Observations

When cultured at 25° C. on a malt extract agar plate, the colony diameter is 18-25 mm at day 4 and 37-48 mm at day 11. At day 11, the color of the center surface of the colonies is very light yellow and the outside surface is yellowish white. The color of the back of the colonies is bright grey yellow.

When cultured at 25° C. on a potato-glucose-agar plate, the colony diameter is 17-18 mm at day 4 and 27-31 mm at day 11. At day 11, the color of the center surface of the colonies is bright gray-yellow and the outside surface is yellowish white. The color of the back of the colonies is bright gray yellow at the center and yellowish white outside. This strain grows in the range of 13.0-34.5° C., with the optimum growth at around 27.5° C. and in the rage of the pH 3.5-11.2 with the optimum growth at around pH 7.3.

2. Microscopic Observations

When cultured at 25° C. for 2 weeks on a malt extract agar plate, the light-microscopic observations of this strain are as follows:

The hypha, that contains septum, is 1.5-2.5 μm wide, smooth, colorless and well branched. The conidiophore, that does not contain septum, is 3.0-5.0 μm wide and 300-350 μm long, smooth, colorless and no branch. The tip of the conidiophore is round forming a vesicle with diameter 15.0-17.5 μm and bowling-pin shaped phialides (10.0-11.5 μm length) are formed from the vesicle. No metulae are formed and from the phialides, the conidia are formed in chains in endoconidium (phialo-type). The phialo-type conidium is a single cell, light green color, globular or semi-globular shaped with smooth surface and with a diameter 2.0-3.0 μm. In this strain only anamorph as described above is observed but not the teleomorph.

Using above-mentioned characteristics of these strains, the search was carried out to determine the taxonomical position of the strains according to The Genera of Fungi Sporulating in Pure Culture, 2^{nd} ed., Cramer, Vanduz, J. A. von Arx, 1974. It was concluded that both strains belong to the imperfect fungi Aspergillus sp. The present inventers named these strains as Aspergillus species MPC1006 and Aspergilus species MPC1009 and deposited at the National Institute of Advanced Industrial Science and Technology (AIST), International Patent Organism Depositary (IPOD) (Tsukuba Central 6, 1-1-1 Higashi, Tsukuba, Ibaraki, Japan) with accession numbers FERM BP-7899 (Date of Deposition: Feb. 18, 2002) and FERM BP-7900 (Date of Deposition: Feb. 18, 2002), respectively.

Another strain, MPC1005, which also represents the strains that produce the compound of the present invention and was isolated from soil, has following characteristics:

1. Macroscopic Observations

When cultured at 25° C. on a malt extract agar plate, the colony diameter is 36-39 mm at day 7 and 73-76 mm at day 11. At day 11, the color of the center surface of the colonies is white and the outside surface is yellowish white. The color of the back of the colonies is light yellow at the center and the outside is very light yellow outside.

When cultured at 25° C. on a potato-glucose-agar plate, the colony diameter is 50-55 mm at day 7 and 75-78 mm at day 11. At day 11, the color of the center surface of the colonies is yellowish white and the outside surface is white. The color of the back of the colonies is dark reddish yellow at the center and very light yellow outside. This strain grows in the range of 13.3-38.6° C., with the optimum growth at around 33.0° C. and in the rage of the pH 3.7-9.4 with the optimum growth at around pH 7.2.

2. Microscopic Observations

When cultured at 25° C. for 4 days on a malt extract agar plate, the light-microscopic observations of this strain are as follows:

The hypha, that contain septum, is 0.5-3.0 μm wide, smooth, colorless and well branched.

When cultured at 25° C. for more than 2 months on both kinds of agar plate described in 1, neither anamorph nor teleomorph was observed.

3. Other Characteristics

The partial nucleotide sequence (1228 bp) of the 18S ribosomal DNA (18S rDNA) of this fungus is shown in the sequence 1.

Using above sequence and known fungi 18S rRNA or 18S rDAN sequences, the molecular phylogenetic analyses were carried out by the neighbor joining method [Program Name: CLUSTAL W, Bulletin of Japanese Society of Microbial Ecology 10:119 (1995)] and the result indicated that this fungus belongs to Phylum Ascomycota, Class Pyrenomycetes and is closely related to Order Xylariales.

From these results, the present inventors named this strain as Xylariales filamentous fungus MPC1005 and deposited at the National Institute of Advanced Industrial Science and Technology (AIST), International Patent Organism Depositary (IPOD) (Tsukuba Central 6, 1-1-1 Higashi, Tsukuba, Ibaraki, Japan) with accession number FERM BP-7980 (Date of Deposition: Mar. 26, 2002).

Still further, the transformations of each functional groups in the Compounds (I), (II), (IIIa), (IIIb), (Va), (Vb), (VI) and the raw material compounds, and in the substituents of those, can be carried out not only by the methods described above but also by other methods that are used generally in synthetic organic chemistry, such as oxidation, reduction, hydrolysis, and other well known techniques [for example, methods described in Comprehensive Organic Transformations, second edition, by R. C. Larock, (John Wiley and Sons Inc) (1999)]. It is also possible to change the order of the reaction process.

The intermediates and the desired compounds in each above-described production process can be isolated and purified by subjecting them to purification methods usually used in synthetic organic chemistry, for example, filtration, extraction, washing, drying, concentration, recrystallization, various chromatography, etc.

Although the Compounds (I), (II), (IIIa), (IIIb), (Va), (Vb) or (VI) may have the stereoisomers such as geometric isomers and optical isomers, all the possible isomers and their mixtures shall be included in these compounds.

When it is needed to obtain salts of the non-peptide compounds, Compound (I), (II), (IIIa), (IIIb), (Va), (Vb) or (VI) used in the present invention, they can be purified as they are if they can be obtained as salts thereof. If they are obtained as their free forms, the non-peptide compounds, Compound (I), (II), (IIIa), (IIIb), (Va), (Vb) or (VI) used in the present invention, can be dissolved or suspended in a suitable solvent, neutralized by adding acid or base and then isolated and purified.

The non-peptide compounds, Compound (I), (II), (IIIa), (IIIb), (Va), (Vb) or (VI) used in the present invention, and pharmaceutically acceptable salts thereof may exist as adducts with water or various solvents. These adducts are also included in the above-described compounds or pharmaceutically acceptable salts thereof.

Examples of the compounds obtained by the present invention are shown in Table 1-3 as Compounds (Va), (Vb) or (VI).

TABLE 1

| | |
|---|---|
| | (VI) |
| 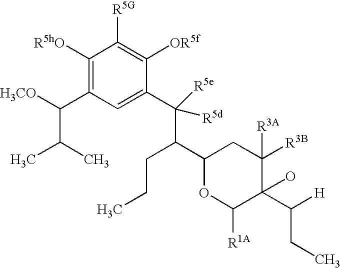 | |

| Compound | R^{1A} | R^{3A}, R^{3B} | R^{5f}, R^{5h} | R^{5d}, R^{5e} | R^{5G} |
|---|---|---|---|---|---|
| 2 | —OH | —OH, H | H | ═O | —CH₂OH |
| 3 | —OCOCH₃ | —OCOCH₃, H | —COCH₃ | ═O | —CH₂OCOCH₃ |
| 4 | —OH | —OH, H | H | H, —OH | —CH₂OH |
| 6 | —OH | —OH, H | H | ═O | —CH═NOCH₃ |
| 14 | —OH | —OH, H | H, CH₃ | ═O | —CH₂C (═O)CH₃ |
| 15 | —OH | ═O | H | ═O | —CHO |

Compound (Va)
Compound 16

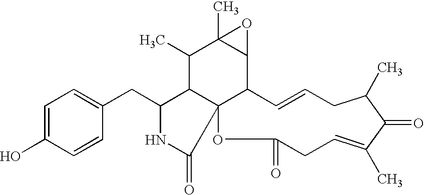

Compound 17

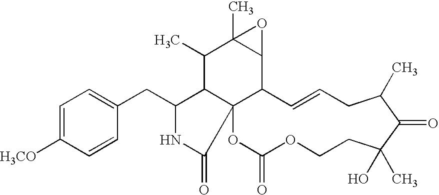

TABLE 3
Compound (Vb)
Compound 18

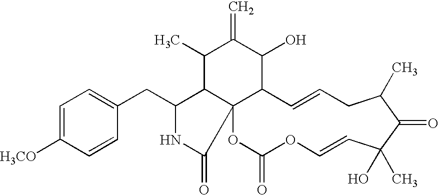

Compound 19

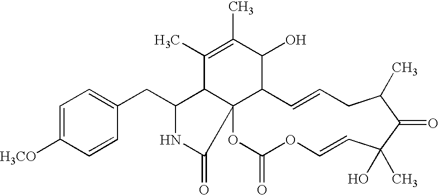

Compound 20

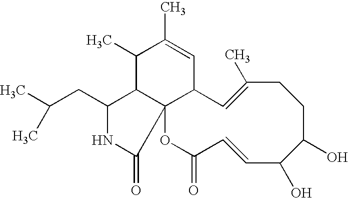

Compound 21

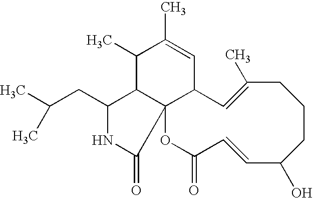

Not only the compounds shown in Tables 1-3 but also other compounds are used in the present invention, and other examples are shown in Tables 4-7 as Compound (I), (II), (IIIa) or (IIIb).

TABLE 4

| | |
|---|---|
| | (I) |
| 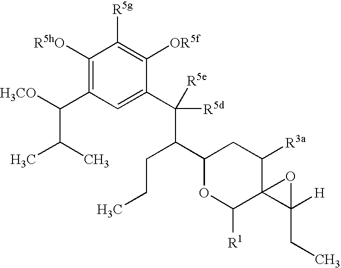 | |

| Compound | R¹, R^{3a} | R^{5f}, R^{5h} | R^{5d}, R^{5e} | R^{5g} |
|---|---|---|---|---|
| 1 (UCS15A) | —OH | H | ═O | —CHO |
| 5 | —OH | H | ═O | —CH═NNHCH₃ |

TABLE 5

| | |
|---|---|
| | (II) |
| 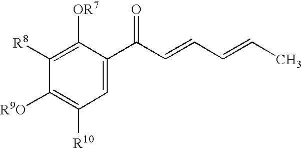 | |

| Compound | R⁷ | R⁸ | R⁹ | R¹⁰ |
|---|---|---|---|---|
| 7 | H | H | H | H |
| 8 | H | —CH₃ | H | H |
| 9 | H | —CH₃ | H | —CH₃ |
| 10 | H | H | —CH₃ | H |
| 11 | —CH₃ | H | —CH₃ | H |

TABLE 6

| | |
|---|---|
| | (IIIa) |
| 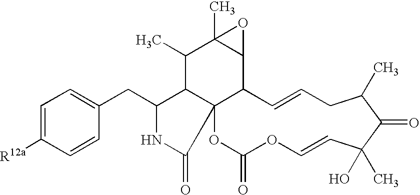 | |

| Compound | R^{12a} |
|---|---|
| 12 (Mer-WF1726) | —OCH₃ |
| 13 (Cytochalasin E) | H |

TABLE 7
Compound (IIIb)
Compound 22

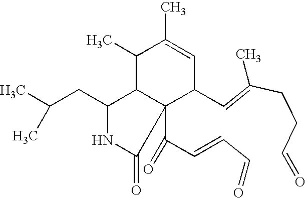

Compound 23

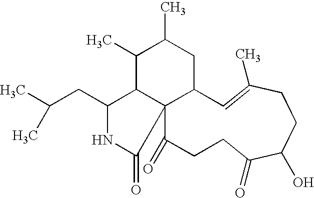

Compound 24 (Aspochalasin A)

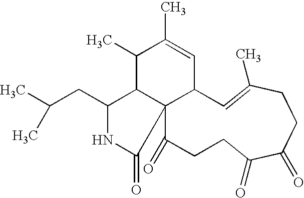

Compound 25 (Aspochalasin B)

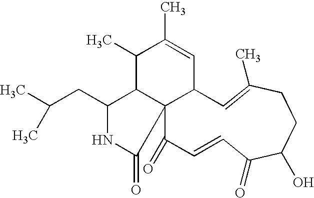

Compound 26 (Aspochalasin C)

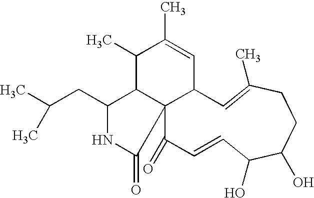

Compound 27 (Aspochalasin D):
isomer of Compound 26

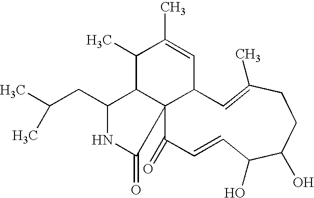

Compound 28 (Aspochalasin E)

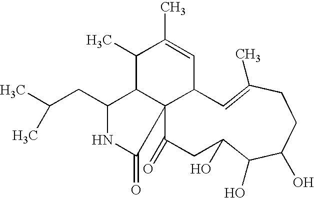

Next, the pharmacological activity of the typical Compound (I), (II), (IIIa), (IIIb), (Va), (Vb) or (VI) is explained concretely with test examples.

### TEST EXAMPLE 1

### SH3 Domain Binding Inhibition Test 1

(1) Treatment of Proteins Containing the Proline-Rich Sequences with the Compounds.

The recombinant human Sam68 containing the 2 proline-rich sequences at the C terminal out of 5 proline-rich sequences in Sam68 containing proline-rich sequences (Sam68ΔC) [Sam68(331-443): made by Santa Cruz Co.], 0.25 μg was added to 1 mL of RSB buffer (10 mmol/L Tris-HCl, pH 7.6, 10 mmol/L NaCl, 1.5 mmol/L MgCl₂), which is a hypotonic buffer solution containing 0.1% BSA (bovine serum albumin) [bovine serum albumin (F-V): made by Nakaraitesk] to prepare a Sam68ΔC solution. The compound was dissolved in dimethylsulfoxide (DMSO) to prepare a 10 mmol/L solution in DMSO. The solution in DMSO was added to the Sam68ΔC solution at final concentrations of 20 and 100 ml/L, respectively. The same amount of DMSO was added to each sample. The compound was subjected to the reaction with Sam68ΔC by incubating the mixture at 37° C. for 6 hours with rotation by a roller culture machine.

(2) Treatment of Proteins Containing the SH3 Domain with the Proteins Containing the Proline-Rich Sequences Treated with the Compounds.

The Sam68ΔC solution treated with the compound, which was obtained in (1), was applied to 5 μL of Fyn-SH3-agarose beads (Fyn-SH3 beads) [Fyn(85-139) AC; made by Santa Crutz Co.], which was a mouse recombinant protein of the Fyn SH3 domain containing the SH3 domain bound to agarose beads. The binding reaction of the Sam68ΔC reacted with the compounds with Fyn-SH3 beads containing the SH3 domain were carried out at 4° C. for 16 hours with slow rotation by the roller culture machine. After the reaction, a resulting complex of Sam68ΔC and Fyn-SH3 beads was precipitated by centrifugation at 50 ppm for 3 minutes at 4° C., and then 1 mL of the TritonX/Np40 extraction buffer containing detergents [50 mmol/L Tris-HCl buffer, pH 7.4, 150 mmol/L NaCl, 1 mmol/Lethylenediaminetetraacetic acid (EDTA), 1% TritonX-100, 0.5% Nonidet P-40] was added thereto. The mixture was rotated slowly at 4° C. for 10 minutes by the roller culture machine, and precipitated and washed twice in a similar manner to that as described above. To the complex of Sam68ΔC and Fyn-SH3 beads washed above, 30 μL of Lemery's sample buffer [Protein Chemistry I-Isolation and Purification, Tokyo Kagaku Dojin, p. 211 (1976)] was added, and then the mixture was mixed well and heated at 100° C. for 10 minutes. After precipitating the beads at 1000 ppm for 5 minutes at 25° C. by a centrifuge, the supernatant was subjected to 10% polyacrylamide gel electrophoresis.

(3) Detection of Sam68ΔC Bound to the Fyn-SH3 by Western Blotting.

Sam68ΔC in the supernatant was detected by Western blotting as follows. First, the resulting gel performed the electrophoresis, which was obtained in (2), was blotted onto a nitrocellulose membrane with pore size of 0.45 μm [Protran; made by Schleicher and Schuell Co.]. After non-specific binding of the membrane was blocked by incubating the membrane at 4° C. overnight in phosphate buffer (PBS) containing 0.25% gelatin and 0.2% Tween-20 (PBS-TG), the membrane reacted with mouse anti GST antibody conjugated with horse radish peroxidase (HRP) [GST (B-14) HRP; made by Santa Crutz Co.], which was diluted 1000 times with PBS-TG, for 2 hours. After washing with PBS containing 0.2% Tween 20 (PBS-T), the detection was carried out by the chemo-luminescence reaction using ECL reagent (Amersham Pharmacia Biotech Co.).

The results are shown in FIGS. 1-3. It was confirmed that the amount of Sam68ΔC bound to Fyn-SH3 beads in the supernatant was decreased by addition of the Compound (I), (II), (IIIa) or (VI) depending on the concentration of Compounds.

### Test Example 2

### SH3 Domain Binding Inhibition Test 2

(1) Preparation of Cell Extracts

HCT116 cells that derived from human colon cancer (ATCC No. CCL-247) were grown in 100 mm cell culture petri dishes using McCoy's 5A modified medium (GIBCO) supplemented with 10% fetal bovine serum at 37° C. in the 5% CO₂ incubator. Compound 1 was added to HCT116 cells in 2 petri dishes cultured for 2 days at final concentrations of 0.5, 1, 2, 2.5, 10, 20 and 30 mmol/L. Compound 1 was diluted with DMSO beforehand and the same amount of DMSO was added to each sample. Compound 1 adding HCT116 cultures were incubated in the 5% CO₂ incubator for 2 hours to carry out the reaction.

HCT116 cells in 2 dishes reacted with Compound 1 were harvested with the medium, and 1 mL of the TritonX/Np40 extraction buffer containing a surfactant added thereto. The medium was mixed well, and stirred with slow rotation by the roller culture machine at 4° C. for 45 minutes to extract intracellular proteins. The cell extracts were centrifuged at 15,000 rpm for 30 minutes at 4° C. and the supernatants were recovered.

(2) Immuno-Precipitation of SH3 Domain Binding Protein Complexes by Antibodies.

The cell extracts treated with the compounds, which was obtained in (1), was mixed with 3 μg of anti-Src antibody (made by Santa Crutz Co.), 3 μg of anti-PLCγ antibody (made by Santa Crutz Co.), or 3 μg of anti-Cortactin antibody (made by Santa Crutz Co.) and incubated at 4° C. for 16 hours with gentle rotation by the roller culture machine. After reacting with antibodies, 30 μL of protein A/G bound agarose beads was added to each SH3 domain binding protein complex and the mixture was incubated at 4° C. for 1 hour with slow rotation by the roller culture machine. The SH3 domain binding protein complex was precipitated at 50 ppm for 3 minutes, suspended into the Triton X/Np40 extraction buffer containing a surfactant, and rotated slowly by the roller culture machine. The same washing and precipitating procedures were repeated three times. To the each washed SH3 domain binding protein complex, 30 μL of Lemery's sample buffer [Protein Chemistry I-Isolation and Purification, Tokyo Kagaku Dojin, p. 211 (1976)] was added, and the mixture was mixed well and heated at 100° C. for 10 minutes. After precipitating the beads at 1000 ppm for 5 minutes at 25° C., the supernatant was subjected to 7.5% polyacrylamide gel electrophoresis.

(3) Detection of Proteins Containing the Proline-Rich Sequences Bound to Proteins Containing the SH3 Domain by Western Blotting.

Protein containing the proline-rich sequences bound to protein containing the SH3 domain was detected by Western blotting as follows. First, the resulting gel performed the electrophoresis, which was obtained in (2), was blotted onto a nitrocellulose membrane with pore size of 0.45 μm [Protran; made by Schleicher and Schuell Co.]. After blotting, non-specific binding of the membrane was blocked by incubating the membrane at 4° C. overnight in PBS-TG. Rabbit anti Sam68, rabbit anti Sos and rabbit anti ZO1 antibodies were used for detecting Sam68, Sos and ZO1, respectively. The each antibody was diluted 1000 times with PBS-TG and reacted with the membrane for 2 hours. After washing with PBS-T, the membrane was reacted with HRP conjugated anti rabbit antibody (made by Amersham), which was diluted 4000 times, for 1 hours. After washing with PBS-T, the detection was carried out by the chemo-luminescence reaction using ECL reagent (Amersham•Pharmacia•Biotech Co.).

The results are shown in FIGS. 4-7. It is confirmed that the amount of protein containing the proline-rich sequences bound to protein containing the SH3 domain was decreased by addition of the compound depending on the concentration of Compounds. Namely, FIG. 4 shows the amount of Sam68 bound to Src, FIG. 5 shows the amounts of Sam68 bound to PLCγ and Grb2, respectively, FIG. 6 shows the amount of SosI bound to Grb2, and FIG. 7 shows the amount of ZO1 bound to Cortactin.

### TEST EXAMPLE 3

### SH3 Domain Binding Inhibition Test 3

(1) Treatment of Nef Containing the Proline-Rich Sequences and Lyn Containing the SH3 Domain with the Compounds.

A mixed solution of Nef and Lyn was prepared by adding 0.3 μg of recombinant Nef containing the proline-rich sequences which was derived from HIV [Nef (3-190); made by CORTEX Co.] and 1 μg of purified bovine Lyn [Lyn; made by upstate Biotechnology Co.] to 1 mL of RSB (10 mmol/L Tris-HCl, pH 7.6, 10 mmol/L NaCl, 1.5 mmol/L MgCl2), which is a hypotonic buffer solution, containing 0.1% BSA (bovine serum albumin). The compound was dissolved in DMSO to prepare a 10 mmol/L solution in DMSO. The solution of compound in DMSO was added to the mixed solution of Nef and Lyn obtained above at final concentrations of 1, 2, 5 and 10 μmol/L, respectively. The compounds were diluted with DMSO beforehand so that the same amount of DMSO was added to each sample. The mixtures of Nef and Lyn adding the compound were rotated at 4° C. for 16 hours by the roller culture machine.

(2) Immuno-Precipitation of SH3 Domain Binding Protein Complexes by Antibodies.

The mixture of Nef and Lyn treated with the compound, which was obtained in (1), was mixed with lug of anti Lyn antibody (made by Santa Crutz Co.) and incubated at 4° C. for 16 hours with slow rotation by the roller culture machine. After reacting with antibody, 10 μL of protein A/G bound agarose beads was added SH3 domain binding protein complex and incubated at 4° C. for 1 hour with slow rotation by the roller culture machine. The SH3 domain binding protein complex was precipitated at 35 ppm for 3 minutes, suspended into 1 mL of the Triton X/Np40 extraction buffer containing of a surfactant, and rotated slowly for 10 minutes by the roller culture machine. The same washing and precipitating procedures were repeated three times. To the washed Nef/Lyn SH3 domain binding protein beads complex, 30 μL Lemery's sample buffer [Protein Chemistry I-Isolation and Purification, Tokyo Kagaku Dojin, pp211 (1976)] was added, and the mixture was mixed well and heated at 100° C. for 5 minutes. After precipitating the beads at 1000 rpm for 5 minutes at 25° C., the supernatant was subjected to 7% polyacrylamide gel electrophoresis.

(3) Detection of Nef Containing the Proline-Rich Sequence Bound to Lyn Containing the SH3 Domain by Western Blotting.

Nef containing the proline-rich sequence in Nef/Lyn complex in the supernatant, which was obtained above, was detected by Western blotting as follows. First, the resulting gel performed the electrophoresis, which was obtained in (2), was blotted onto a nitrocellulose membrane with size of 0.45 μm [Protran; made by Schleicher and Schuell Co.]. After blotting, non-specific binding of the membrane was blocked by pouring PBS-TG on the membrane. A anti β galactosidase antibody (anti β-gal antibody) was used for detecting Nef, because the recombinant Nef protein used here was fused with β galactosidase. The rabbit anti β-gal antibody was diluted 1000 times with PBS-TG and reacted with the membrane for 2 hours. After washing with PBS-T, the membrane was reacted with a HRP conjugated anti rabbit antibody (made by Amersham), diluted 4000 times with PBS-TG, for 1 hour. After washing with PBS-T, the detection was carried out by the chemo-luminescence reaction using ECL reagent (Amersham•Pharmacia•Biotech Co.).

The results are shown in FIG. 8. It is confirmed that the amount of Nef containing the proline-rich sequence bound to Lyn protein containing the SH3 domain was decreased by the addition of the compounds depending on the concentration of Compounds.

### TEST EXAMPLE 4

### Calculation of Inhibition Ratio of the Compound Against the SH3 Domain Binding.

The inhibition ratios of the compound against the SH3 domain binding were calculated as follows based on the plot of the results of the Test Example 1. The intensity of the chemo-luminescent bands of Fyn-SH3 and Sam68ΔC developed on a film was measured by a gel-scanner (TYOBO). The relative intensity of Sam68ΔC band against the intensity of Fyn-SH3 band was obtained by dividing the Sam68ΔC band intensity by the Fyn-SH3 band intensity. The ratio of the Fyn-SH3 band intensity to the Sam68ΔC band intensity was set to be 100% when no compound was added. The inhibition ratio of the compound against the SH3 domain binding is defined as the ratio of decrease of Sam68ΔC band intensity after the addition of the compound. The results are shown in Table 8.

TABLE 8
The inhibition ratio (%) of the compound against SH3
domain binding

| | Concentration of the compound (μmol/L) | | | | |
|---|---|---|---|---|---|
| Compound | 0 | 20 | 60 | 100 | 150 |
| 1 | 0 | | 0 | | 46 |
| 2 | 0 | 34 | | 95 | |
| 3 | 0 | 75 | | 100 | |
| 4 | 0 | 47 | | 85 | |
| 5 | 0 | 77 | | 100 | |
| 6 | 0 | 28 | | 37 | |
| 7 | 0 | | | | 82 |
| 8 | 0 | | | | 77 |
| 9 | 0 | | | | 90 |
| 10 | 0 | | | | 71 |
| 11 | 0 | | | | 84 |
| 12 | 0 | 39 | | 88 | |
| 13 | 0 | 0 | | 21 | |
| 14 | 0 | | | 80 | |
| 15 | 0 | | | 39 | |
| 16 | 0 | | 67 | | 78 |
| 17 | 0 | | | | 36 |
| 18 | 0 | | 16 | | 67 |
| 19 | 0 | | | | 40 |
| 20 | 0 | | 92 | | 100 |
| 21 | 0 | | 64 | | 100 |
| 22 | 0 | | 91 | | 100 |
| 23 | 0 | | | | 82 |
| 24 | 0 | | 62 | | 80 |
| 25 | 0 | | 87 | | 100 |
| 26 | 0 | | 92 | | 100 |
| 27 | 0 | | 100 | | 100 |
| 28 | 0 | | 31 | | 73 |

Results of Test Example 1-4 demonstrated that Compounds (I), (II), (IIIa), (IIIb), (Va), (Vb) and (VI) had superior SH3 domain binding inhibitory activity.

### TEST EXAMPLE 5

### HIV-1 Growth Inhibition Test

(1) HIV-1 Infection and Growth Inhibition Experiment.

MT2 cells (T cell strain) were suspended in RPMI1640 medium (GIBCO No.26140-076) supplemented with 10% fetal calf serum (GIBCO, No.26140-079) at 2×10⁵ cells/mL and distributed into the each well of 24 well plates (24 well plate: Coster No. 3526) by 1 mL per well. HIV-1 LAI strain {pLAI that contains DNA clone of the whole genome [GeneBank accession No. K02013 Virology 185:661 (1991)] was transfected to HeLa cells by using FuGENE6 Transfection Reagent (Roche Co. No. 1814443), incubated for 2 days and the supernatant was used.} was inoculated to each well at a final concentration of 500 cpm/μL and incubated at 37° C. overnight (virus infection). The compound or PP2 (protein phosphatase 2:4-amino-5-(4-chrolophenyl)-7-(tert-butyl)-pyrazolo[3,4-d]pyrimidine, the inhibitor of tyrosine phosphrylation of Src: control, made by Alexs Co.) was added thereto at final concentrations of 1, 5, 10, 25 and 50 mmol/L (drug addition), respectively. At 3, 5, 7 and 9 days after the virus infection, 50 μL of the culture supernatant was harvested and stored at −80° C. until the measurement. The positive control wells were infected and the compound was not added thereto, and the negative control wells were not infected and the compound was not added thereto. The same three experiments were repeated in these tests.

(2) Measurement of HIV-1 Amount

HIV-1 was determined by the RT assay (Reverse Transcriptase sassy) according to the method described in J. Virol., Vol. 38, p. 239 (1981) with a minor modification.

To 5 μL of the culture supernatant obtained in (1), 25 μL of RT cocktail {50 mmol/L Tris-HCl pH 7.5, 50 mmol/L EDTA, 75 mmol/L KCl, 5 mmol/L MgCl2, 5 μg/mL Poly(A) [Pharmacia, No. 27-4110-01], 1.6 μg/mL oligo(dT)12-18 [Pharmacia, No. 27-7858-01], 5 mmol/L DTT (dithiothreitol), 0.05% NP-40 and 10 PCi/ml [α-³²P]dTTP} was added, and the mixture was incubated at 37° C. for 2 hours. After the reaction, 6 μL of the reaction mixture was spotted on a DEAE filter mat [Perkin Elmer, No. 1450-522] to remove un-reacted [α-³²P] dTTP and then the resulting filtermat was immersed in MeltiLex A melt-on scintillator. The amount of HIV-1 was measured as RT activity count (cpm/μL) with a MICROBRTA PLUS liquid scintillation counter (Perkin Elmer).

The results are shown in FIGS. 12-14, it is confirmed that the amount of HIV-1 increased by addition of the control pp2. The results are shown in FIGS. 9-11, it is confirmed that the increase in the amount of HIV-1 was suppressed by the addition of the compound depending on the concentration of the compound. Namely, these results indicate that the addition of the compound inhibited the growth of HIV-1.

### TEST EXAMPLE 6

### Cyto-Toxicity Test

MT2 cells (T cell strain) were suspended in RPMI1640 medium (GIBCO, No. 26140-076) supplemented with 10% fetal calf serum (GIBCO, No. 26140-079) at 2×10⁵ cells/mL and distributed into each well of 96 well plates [96 well plate: Coster] at 100 μL per well. The compound was added to each well and incubated for 3 days. The cultures were observed under a microscope to examine the condition of cells and cells were harvested, stained with 0.4% Trypan blue (SIGMA, T-8154) and live cells were counted. As a result, live cells were not decreased in the presence of 10 μmol/L of Compound 1.

The results of the Test Examples 1-6 suggested that medicaments, which comprise the compounds exhibiting superior SH3 domain binding inhibitory activity as an active ingredient, are effective against various diseases involving the protein-protein interaction mediated by the SH3 domain {for example, AIDS, malignant tumor, allergic diseases, virus diseases, etc. [Biopolymer, Vol. 43, p. 383 (1997) etc.]}.

Although the Compound (I), (II), (IIIa), (IIIb), (Va), (Vb) or (VI), or pharmaceutically acceptable salts thereof can be administered as such, it is generally preferred to offer them in the form of various pharmaceutical preparations. These pharmaceutical preparations can be used in animals or humans.

The pharmaceutical preparations of the present invention can contain, as an active ingredient, the Compound (I), (II), (IIIa), (IIIb), (Va), (Vb) or (VI), or pharmaceutically acceptable salts thereof alone or in combination with any other active ingredients for the treatment of different diseases. These pharmaceutical preparations are produced by any of the suitable methods well known in the technical field of pharmaceutics by mixing the active ingredient with one or more pharmaceutically acceptable carriers.

It is preferred to use the administration route that is most effective for the treatment, for example oral administration or non-oral administration such as intravenous administrations.

The pharmaceutical preparations can be in the forms of tablets, powders, granules, syrups, injections, etc.

The liquid products such as syrups for oral administration can be made by using water; sugars such as sucrose, sorbitol and fructose; glycols such as polyethylene glycol and propylene glycol; oils such as sesame oil, olive oil and soy bean oil; preservatives such as p-hydroxybenzoic acid esters; and flavors such as strawberry flavor and peppermint. Tablets, powders, granules, etc. can be produced by using excipients such as lactose, glucose, sucrose and mannitol; disintegrats such as starch and sodium alginate; lubricants such as magnesium stearate and talc; binders such as polyvinyl alcohol, hydroxypropyl cellulose and gelatin; surfactants such as fatty acid esters; and plasticizers such as glycerin; etc.

The pharmaceutical preparations suitable for non-oral administration are preferably sterilized solutions of active compounds that are isotonic with the blood of the recipients. For example, the injections are prepared by using carriers that are made from solution of salts, solution of glucose or a mixture of solution of salts and solution of glucose.

In these non-oral agents, the same diluents, preservatives, flavors, excipients, disintegrators, lubricants, binders, detergents, and plasticizers used in the oral agents, can be added alone or in combination of more than one as supplements.

The dosage and the frequency of administration of the Compound (I), (II), (IIIa), (IIIb), (Va), (Vb) or (VI), or pharmaceutically acceptable salts thereof vary depending on the rout of the administration, the age and body weight of the patients, and the nature or the seriousness of the symptoms to be treated. The oral administration dosage can be ordinarily 0.01 mg-1 g, preferably 0.05-50 mg, once or several times a day for an adult. In the case of non-oral administration such as intravenous administration, the dosage can be 0.001-100 mg, preferably 0.01-10 mg, once or several times a day for adults. However, the dosage and the frequency of the administration can be varied depending on the various conditions described above.

### BRIEF DESCRIPTION OF THE FIGURES

FIG. 1 shows the results of in vitro assay for the Sam68ΔC and Fyn-SH3 binding inhibition when Compounds (I) and (VI) were added at different concentrations. The numbers on top of the lanes demonstrate the compound number and the concentration (μmol/L) of the compound added to the solution of Sam68ΔC. The name of each blot is shown in the left.

FIG. 2 shows the results of in vitro assay for the Sam68ΔC and Fyn-SH3 binding inhibition when Compounds 1 and (IIIa) were added at different concentrations. The numbers on top of the lanes demonstrate the compound number and the concentration (μmol/L) of the compound added to the solution of Sam68ΔC. The name of each blot is shown in the left.

FIG. 3 shows the results of in vitro assay for the Sam68ΔC and Fyn-SH3 binding inhibition when the Compounds (II) and (IIIa) were added at different concentrations. The numbers on top of the lanes demonstrate the compound number and the concentration (Pmol/L) of the compound added to the solution of Sam68ΔC. The name of each blot is shown in the left.

FIG. 4 shows the results of the assay of the intracellular Src and Sam68 binding inhibition when the Compound 1 was added at different concentrations. The numbers on top of the lanes demonstrate the concentration (μmol/L) of the Compound 1 added to the HCT116 cells. The name of each blot is shown in the left.

FIG. 5 shows the results of the assay of the intracellular PLCγ and Sam68 binding inhibition when Compound 1 was added at different concentrations. The numbers on top of the lanes demonstrate the concentration (μmol/L) of Compound 1 added to the HCT116 cells. The name of each blot is shown in the left.

FIG. 6 shows the results of the assay of the intracellular Grb2 and Sos1 binding inhibition when Compound 1 was added at different concentrations. The numbers on top of the lanes demonstrate the concentration (μmol/L) of Compound 1 added to the HCT116 cells. The name of each blot is shown in the left.

FIG. 7 shows the results of the assay of the intracellular Cortactin and Z1 binding inhibition when Compound 1 was added at different concentrations. The numbers on top of the lanes demonstrate the concentration (μmol/L) of Compound 1 added to the HCT116 cells. The name of each blot is shown in the left.

FIG. 8 shows the results of in vitro assay for the Nef and Lyn binding inhibition when Compounds 1 and 3 were added at different concentrations. The numbers on top of the lanes demonstrate the compound number and the concentration (μmol/L) of each Compound added to the mixed solution of Nef and Lyn. The name of each blot is shown in the left.

FIGS. 9-11 show the results of the HIV-1 growth inhibition assay (RT assay) when Compound 1 was added at different concentrations. Results of three experiments in the same condition are shown. The amount of HIV-1 is shown as the counts of RT assay (cpm/μL) at the left of the axis of ordinates. The time required since the infection of HIV-1 (days) are shown under the axis of abscissas. The plots in the graphs are as follows: □ white square]: positive control, × [cross]: negative control, ● [black circle]: Compound 1 (50 mmol/L), ▴ [black triangle]: Compound 1 (25 mmol/L), ▪ [black square]: Compound 1 (10 mmol/L), ∘ [white circle]: Compound 1 (5 mmol/L), Δ [white triangle]: Compound 1 (1 μmol/L)

FIGS. 12-14 show the results of the HIV growth inhibition assay (RT assay) when PP2 was added at different concentrations. Results of three experiments in the same condition are shown. The amount of HIV-1 is shown as the counts of RT assay (cpm/μl) at the left of the axis of ordinates. The time required since theinfectionofHIV-1 (days) areshownundertheaxisofabscissas. The plots in the graphs are as follows: □ : positive control, × : negative control, ● [black circle]: PP2 (50 mmol/L), ▴ [black triangle]: PP2 (25 mmol/L), ▪ [black square] PP2 (10 μmol/L), ∘ [white circle]: PP2 (5 mmol/L), Δ [white triangle]: PP2 (1 μmol/L)

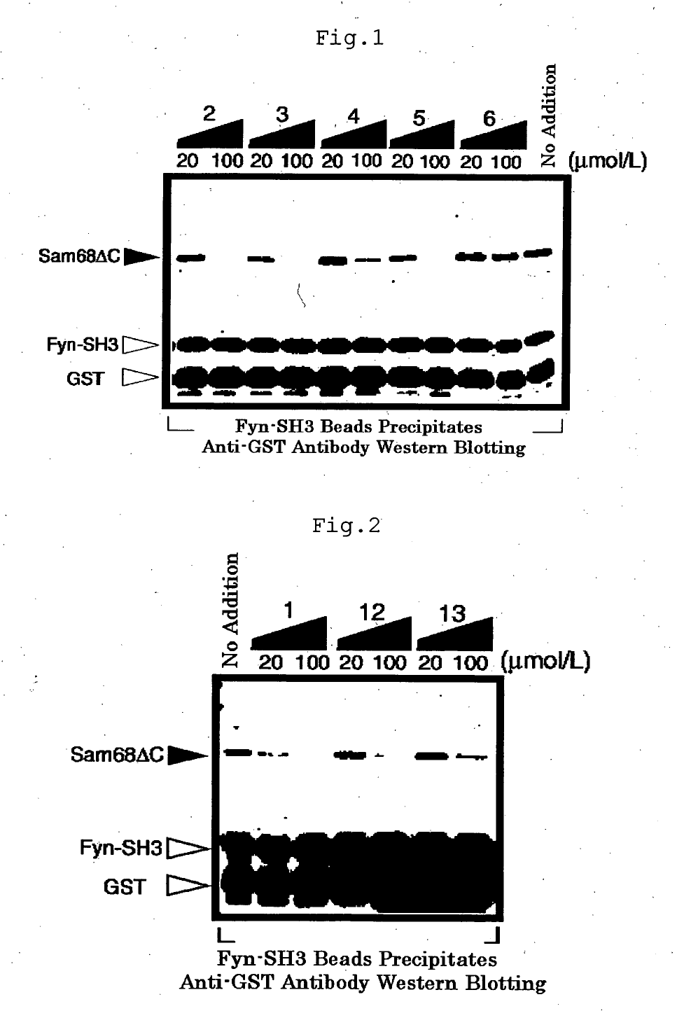

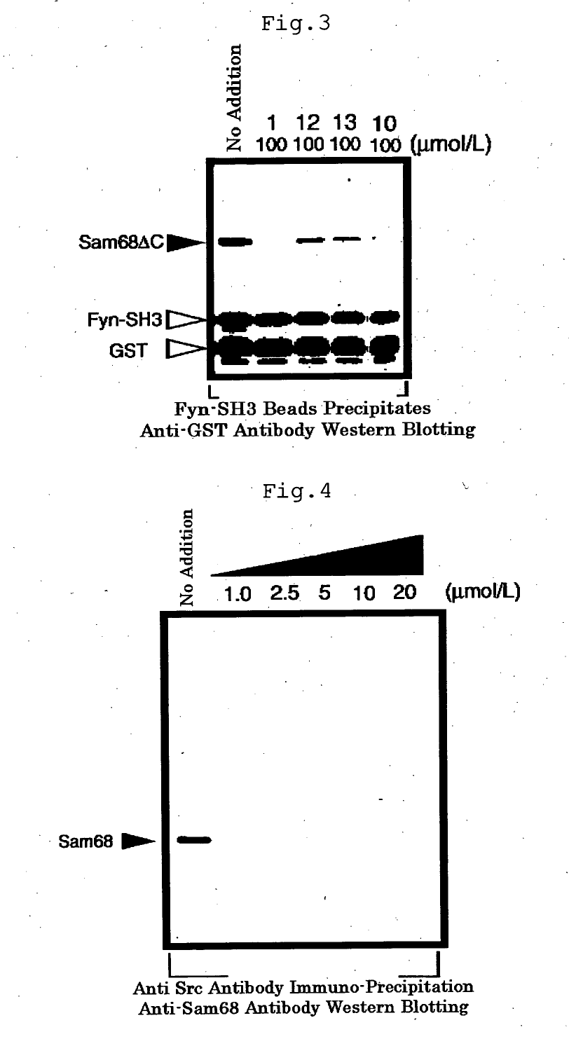

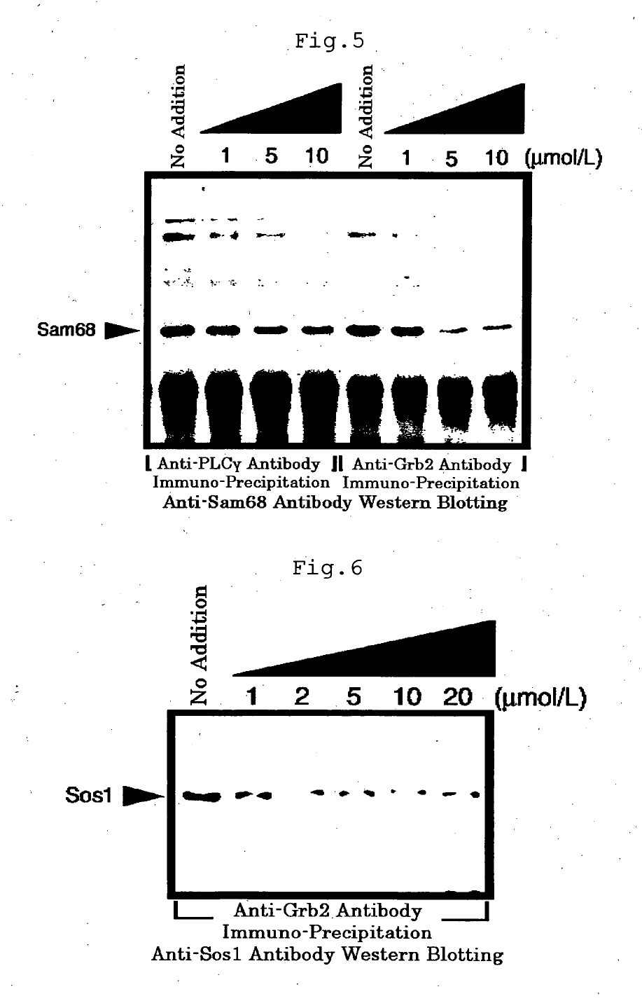

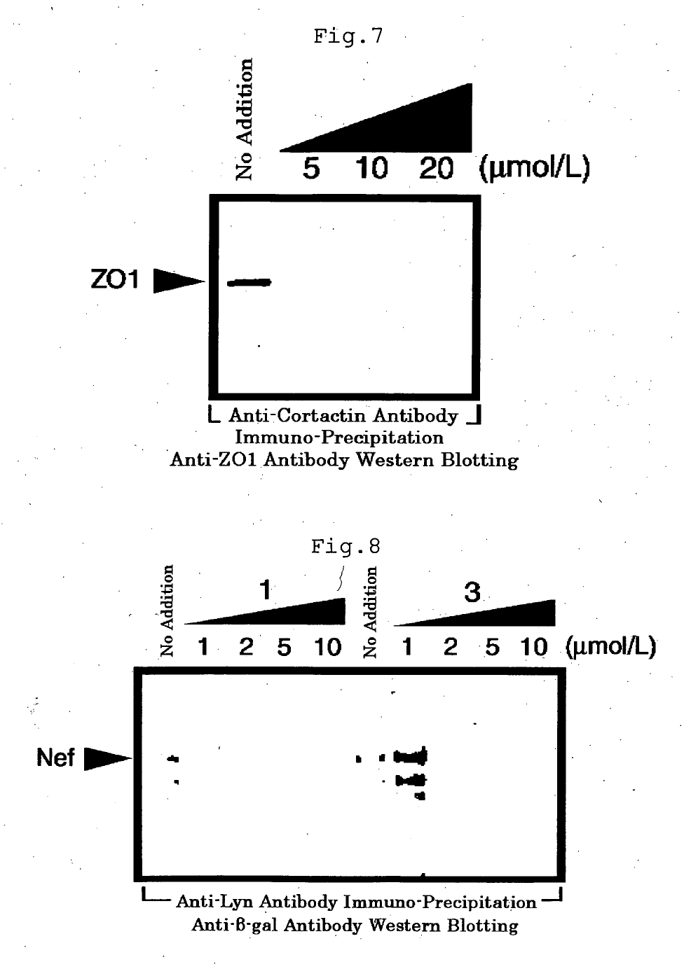

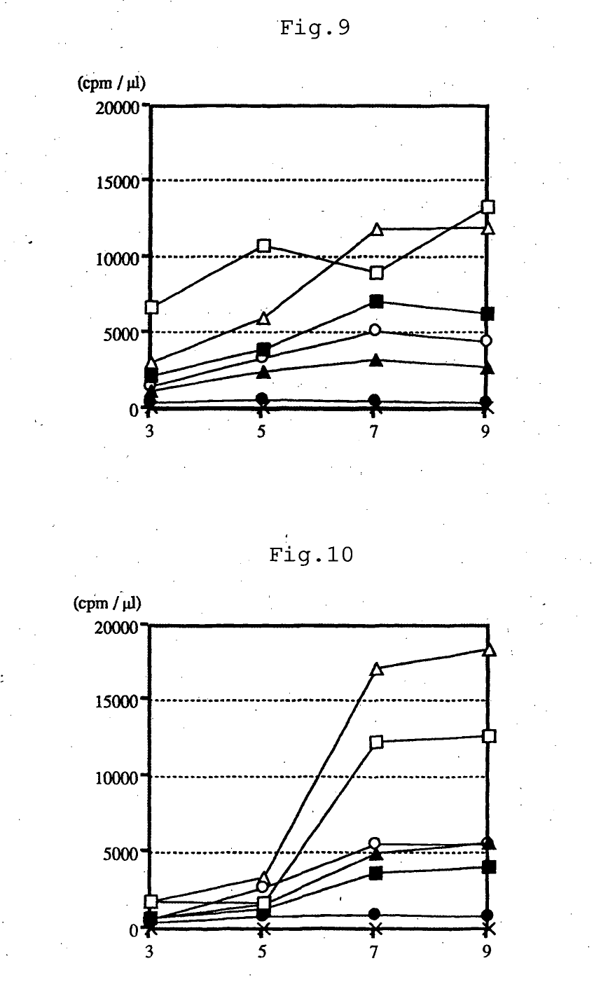

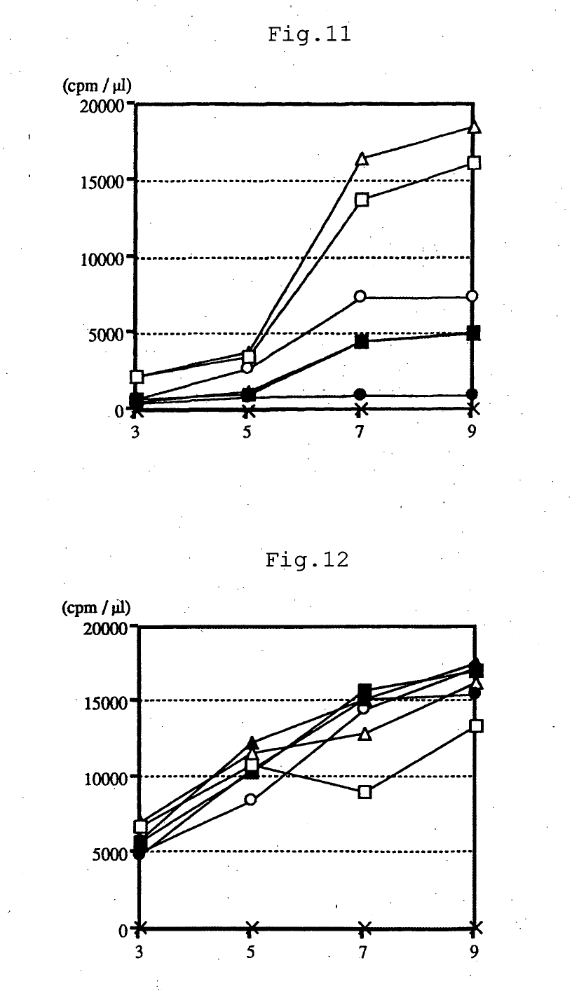

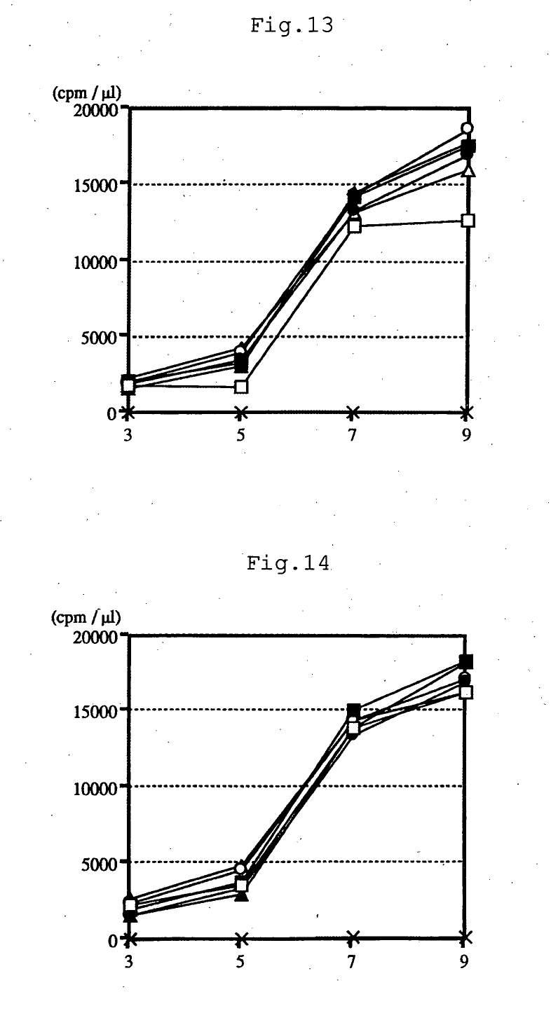

### BEST MODE FOR CARRYING OUT THE INVENTION

The present invention is described in detail below referring to reference examples and examples.

### EXAMPLE 1

### Tablet

Tablets comprising the following composition were prepared by a conventional method. Compound 2 (40 g), lactose (286.8 g) and potato starch (60 g) were mixed, and 10% solution of hydroxypropylcellulose (120 g) was added thereto. After the resulting mixture was kneaded, granulated and dried, the size of the granules was controlled for tablet pressing. The granules were mixed with magnesium stearate (1.2 g) and pressed to make tablets (each tablet contains 20 mg of an active ingredient) by a tablet making machine having a striker of 8 mm diameter (Kikusui Co., Type RT-15).

Formulation: Compound 220 mg

| | | |
|---|---|---|
| Formulation: | Compound 2 | 20 mg |
| | Lactose | 143.4 mg |
| | Potato starch | 30 mg |
| | Hydroxypropylcellulose | 6 mg |
| | Magnesium stearate | 0.6 mg |
| | | 200 mg |

### EXAMPLE 2

### Capsule

Capsules comprising the following composition were prepared by a conventional method. Compound 12 (200 g), Avicel (995 g) and magnesium stearate (5 g) were mixed by the conventional method. The resulting mixture was filled in a hard capsules No. 4 (with a volume of 120 mg per 1 capsule) by a capsule charzer (Zanasi Co., Type LZ-64) to provide capsules (each capsule contains 20 mg of an active ingredient).

| | | |
|---|---|---|
| Formulation: | Compound 12 | 20 mg |
| | Avicel | 99.5 mg |
| | Magnesium stearate | 0.5 mg |
| | | 120 mg |

### Example 3

### Injection

An injection comprising the following composition was prepared by a conventional method. Compound 10 (1 g) was dissolved in refined soybean oil, and refined egg yolk lecithin (12 g) and glycerin for injection (25 g) were added thereto. Injectable distilled water was added to make the total volume 1000 mL, and the resulting mixture was suspended well and emulsified. The resulting dispersion was filter-sterilized with a 0.2 μm disposable membrane filter and dispensed into glass vials at a volume of 2 mL per vial (each vial contains 2 mg of the active ingredient) under the sterile condition to obtain the injection.

| | | |
|---|---|---|
| Formulation: | Compound 10 | 2 mg |
| | Purified soy bean oil | 200 mg |
| | Purified egg yolk lecithin | 24 mg |
| | Glycerin for Injection | 50 mg |
| | Water for Injection | 1.72 mL |
| | | 2.00 mL |

### EXAMPLE 4

### Compound 2

Compound 1 (20 mg; 0.042 mmol) obtained in the Reference Example 1 was dissolved in THF (2 mL) and a 70% solution of sodium bis(2-methoxyethoxy) aluminum hydride in toluene (0.040 mL) was added thereto under ice-cooling. After the mixture was stirred at 0° C. for 20 minutes, a 1 mol/L solution of acetic acid in THF (1 mL) and ethyl acetate (10 ml) were sequentially added to the reaction mixture and extracted. The organic layer was washed with water and 1 mol/L hydrochloric acid. The organic layer was dried over anhydrous sodium sulfate and then concentrated. The resulting residue was crystallized from a mixed solvent of diisopropylether (IPE) and n-hexane to obtain 12.3 mg of Compound 2 (yield 61%).

Compound 2:

¹H-NMR (CDCl₃) δ (ppm): 0.78 (3H, d, J=6.6 Hz), 0.86 (3H, t, J=7.3 Hz), 1.02 (3H, d, J=6.6 Hz), 1.07 (3H, t, J=7.7 Hz), 1.23 (2H, m), 1.52-1.61 (3H, m), 1.79 (2H, m), 1.96-2.01 (2H, m), 2.66 (1H, d, J=2.9 Hz), 3.27 (1H, t, J=6.1 Hz), 3.39 (3H, s), 3.54 (1H, m), 3.92 (1H, d, J=7.7 Hz), 4.16 (1H, m), 4.35 (1H, brt, J=9.9 Hz), 4.87 (2H, s), 4.97 (1H, d, J=2.8 Hz), 7.41 (1H, s), 9.24 (1H, s), 13.4 (1H, s); FAB-MS m/z 505 [M+Na]⁺.

### EXAMPLE 5

### Compound 3

Compound 2 (10 mg; 0.021 mmol) obtained in Example 4 was dissolved in pyridine (0.5 mL), and acetic anhydrate (0.5 mL) was added thereto. The mixture was stirred for five hours at room temperature. After the reaction mixture was concentrated, the resulting residue was crystallized from a mixed solvent of IPE and n-hexane to obtain 4.6 mg of Compound 3 (yield 32%). Compound 3:

¹H-NMR (CDCl₃) b(ppm): 0.83 (3H, t, J=7.2 Hz), 0.84 (3H, d, J=7.2 Hz), 0.93 (3H, d, J=6.8 Hz), 0.99 (3H, t, J=7.5 Hz), 1.21 (2H, m), 1.56 (3H, m), 1.76 (3H, m), 1.95 (1H, m), 2.01 (3H, s), 2.04 (3H, s), 2.13 (3H, s), 2.35 (3H, s), 2.37 (3H, s), 2.99 (1H, t, J=5.9 Hz), 3.18 (3H, s), 3.57 (1H, m), 3.89 (1H, d, J=6.2 Hz), 4.29 (1H, m), 5.03 (2H, s), 5.50 (1H, dd, J=4.8, 12.1 Hz), 5.87 (1H, s), 7.91 (1H, s); FAB-MS m/z 693 [M+H]⁺.

### EXAMPLE 6

### Compound 4

Compound 1 (11 mg; 0.023 mmol) obtained in the Reference Example 1 was dissolved in methanol (1 mL), and sodium borohydride (4.9 mg; 0.13 mmol) was added thereto. The mixture was stirred for 1.5 hours at room temperature. A small quantity of 1 mol/L hydrochloric acid was added to the reaction mixture and extracted with ethyl acetate (10 mL×2). The organic layer was washed with a saturated aqueous sodium chloride solution. The organic layer was dried over anhydrous magnesium sulfate, and then concentrated. The resulting residue was purified by preparative silica gel thin-layer chromatography (chloroform/methanol=10/1) to obtain 6.0 mg of Compound 4 (yield 54%).

Compound 4:

¹H-NMR (CDCl₃) δ(ppm): 0.80 (3H, d, J=6.8 Hz), 0.89 (3H, t, J=7.3 Hz), 1.01 (3H, t, J=7.5 Hz), 1.02 (3H, d, J=7.5 Hz), 1.26 (2H, m), 1.54-1.64 (3H, m), 1.79 (3H, m), 2.00 (1H, m), 2.67 (1H, brs), 2.74 (1H, brs), 2.91 (1H, t, J=6.4 Hz), 2.98 (1H, d, J=2.9 Hz), 3.27 (1H, brs), 3.38 (3H, s), 3.47 (1H, m), 3.72-3.89 (2H, m), 3.96 (1H, t, J=7.5 Hz), 4.18 (1H, brs), 4.88 (3H, brs), 7.41 (1H, s), 9.32 (1H, s), 13.29 (1H, s); FAB-MS m/z 507 [M+Na]⁺.

### EXAMPLE 7

### Compound 6

Compound 1 (10 mg; 0.021 mmol) obtained in the Reference Example 1 was dissolved in a mixed solvent of THF (1 mL) and water (0.5 mL), and o-methylhydroxylamine hydrochloride (22 mg; 0.27 mmol) was added thereto. The mixture was stirred for 4 hours at room temperature. The reaction mixture was diluted with ethyl acetate (10 mL) and extracted after the addition of water. The organic layer was washed with a 1 mol/L hydrochloric acid. The organic layer was dried over anhydrous magnesium sulfate and then concentrated. The resulting residue as crystallized from a mixed solvent of IPE and n-hexane to obtain 5.6 mg of Compound 6 (yield 52%).

Compound 6:

¹H-NMR (CDCl₃) δ (ppm): 0.84 (3H, t, J=7.3 Hz), 0.86 (3H, d, J=6.8 Hz), 0.96 (3H, d, J=6.8 Hz), 1.06 (3H, t, J=7.5 Hz), 1.20 (2H, m), 1.48 (1H, m), 1.58 (1H, s), 1.60 (2H, m), 1.81 (2H, m), 1.99 (2H, m), 2.98 (1H, brs), 3.22 (3H, s), 3.26 (1H, t, J=6.1 Hz), 3.70 (1H, dd, J=7.3, 14.1 Hz), 3.99 (3H, s), 4.17 (1H, td, J=11.2, 5.1 Hz), 4.34 (1H, d, J=6.6 Hz), 4.40 (1H, brt, J=8.3 Hz), 4.97 (1H, d, J=3.5 Hz), 7.83 (1H, s), 8.69 (1H, s), 11.45 (1H, s), 13.75 (1H, s); FAB-MSm/z 532 [M+Na]⁺, 510[M+H]⁺.

### EXAMPLE 8

### Compound 14

Compound 1 (33.5 mg; 0.0698 mmol) obtained in the Reference Example 1 was dissolved in methanol (2 mL), and a 2 mol/L solution of (trimethylsilyl)diazomethane in n-hexane (1 mL) added thereto. The mixture was stirred for 3.5 hours at room temperature. Two drops of acetic acid and ethyl acetate (30 mL) were added to the reaction mixture, and extracted after addition of water. The organic layer was washed with a saturated aqueous sodium chloride solution and dried over anhydrous magnesium sulfate. After the solvent was distilled away, the resulting residue (45.6 mg) was purified by preparative silica gel thin-layer chromatography (n-hexane/ethyl acetate=1/1) to obtain 13.9 mg of Compound 14 (yield 38%).

Compound 14:

¹H-NMR (CDCl₃, 400 MHz) δ(ppm): 12.91 (1H, s), 7.81 (1H, s), 4.97 (1H, d, J=2.4 Hz), 4.41 (1H, m), 4.18 (1H, m), 4.15 (1H, d, J=6.8 Hz), 3.77 (2H, d, J=5.6 Hz), 3.75 (3H, s), 3.72 (1H, m), 3.26 (1H, dd, J=5.9, 7.1 Hz), 3.22 (3H, s), 2.93 (1H, brs), 2.26 (3H, s), 2.01 (1H, m), 1.92 (1H, m), 1.84 (2H, m), 1.58 (3H, m), 1.47 (1H, m), 1.20 (2H, m), 1.06 (3H, t, J=7.6 Hz), 0.98 (3H, d, J=6.6 Hz), 0.86 (3H, t, J=7.3 Hz), 0.81 (3H, d, J=6.8 Hz); ¹³C-NMR (CDCl₃, 100 MHz) δ(ppm): 208.0, 206.3, 164.1, 162.0, 129.2, 125.3, 117.6, 117.4, 94.5, 82.8, 69.7, 62.7, 61.9, 61.8, 59.7, 57.0, 49.9, 39.0, 37.3, 34.6, 32.0, 29.7, 20.7, 20.6, 19.1, 18.3, 14.3, 10.6; FAB-MS m/z 523 [M+H]⁺.

### EXAMPLE 9

### Compound 15

Compound 1 (21.5 mg; 0.0448 mmol) obtained in the Reference Example 1 was dissolved in dichloromethane (2 mL) and 4 Angstrom molecular sieves (30 mg) was added thereto. And then, a solution of 4-methylmorpholine-N-oxide (18.6 mg; 0.159 mmol) in dichloromethane (0.5 mL) and a solution of tetra-n-propyl ammonium perruthenate (3.9 mg; 0.0111 mmol) in dichloromethane (0.5 mL) were added thereto, and the mixture was stirred for 24 hours at room temperature. The reaction mixture was filtered and then was purified by silica gel column chromatography (1% methanol in chloroform solution) to obtain 1.8 mg of Compound 15 (yield 8.4%).

Compound 15:

¹H-NMR (CDCl₃, 500 MHz) δ(ppm): 14.03 (1H, s), 13.01 (1H, s), 10.42 (1H, s), 8.03 (1H, s), 5.20 (1H, d, J=3.2 Hz), 4.84 (1H, m), 4.34 (1H, d, J=6.1 Hz), 3.77 (1H, m), 3.56 (1H, dd, J=5.6, 7.2 Hz), 3.22 (3H, s), 3.01 (1H, d, J=3.2 Hz), 2.68-2.63 (2H, m), 2.00-1.80 (2H, m), 1.79-1.60 (3H, m), 1.26 (2H, m), 1.08 (3H, t, J=7.6 Hz), 0.94 (3H, d, J=6.7 Hz), 0.90 (3H, d, J=6.9 Hz), 0.89 (3H, t, J=7.5 Hz); FAB-MS m/z 479 [M+H]⁺.

### EXAMPLE 10

### Compound 17

Compound 12 (67.3 mg; 0.128 mmol) obtained in Reference Example 9 was dissolved in ethyl acetate (3 mL), and 10% palladium-carbon (50 mg) was added thereto. The mixture was stirred for 4 hours at room temperature under hydrogen atmosphere. After removing the catalyst by filtration, the reaction mixture was purified by preparative silica gel thin-layer chromatography (a mixed solvent of n-hexane and ethyl acetate) to obtain 30.3 mg of Compound 17 (yield 63%).
Compound 17:

¹H-NMR (CDCl₃, 300 MHz) δ(ppm): 7.03 (2H, d, J=8.6 Hz), 6.85 (2H, d, J=8.6 Hz), 6.40 (1H, m), 6.07 (1H, brs), 5.31 (1H, m), 4.86-4.28 (2H, m), 3.79 (3H, s), 3.61 (1H, m), 3.19 (1H, dd, J=5.5, 10.5 Hz), 3.04 (1H, m), 2.80 (1H, d, J=5.5 Hz), 2.76-2.49 (4H, m), 2.36 (1H, m), 2.19 (1H, brd, J=12.5 Hz), 1.71 (1H, brd, J=15.8 Hz), 1.49 (3H, s), 1.23 (2H, m), 1.19 (3H, d, J=6.8 Hz), 1.14 (3H, s), 1.00 (3H, d, J=7.2 Hz); FAB-MS m/z 528 [M+H]⁺.

### EXAMPLE 11

### Compounds 18 and 19

Compound 12 (36.6 mg; 0.0697 mmol) obtained in Reference Example 9 was dissolved in chloroform (2 mL), and a small quantity of p-toluenesulfonic acid hydrate was added thereto. The mixture was stirred for 5 hours at room temperature. The reaction mixture was concentrated and was purified by preparative silica gel thin layer chromatography (a mixed solvent of n-hexane and ethyl acetate) to obtain Compound 18 (26.8 mg; yield 73%) and 19 (4.8 mg; yield 13%), respectively.

Compound 18:

¹H-NMR (CDCl₃, 300 MHz) δ(ppm): 7.04 (2H, d, J=8.6 Hz), 6.84 (2H, d, J=8.6 Hz), 6.59 (1H, d, J=11.7 Hz), 5.81 (1H, brs), 5.72 (1H, dd, J=9.9, 14.9 Hz), 5.63 (1H, d, J=11.7 Hz), 5.39 (1H, brs), 5.37 (1H, m), 5.17 (1H, brs), 3.80 (1H, m), 3.78 (3H, s), 3.35 (1H, m), 3.24 (1H, m), 2.98-2.92 (3H, m), 2.85 (1H, dd, J=3.9, 13.8 Hz), 2.69 (1H, m), 2.57 (1H, dd, J=9.5, 13.8 Hz), 2.15 (1H, brd, J=13.4 Hz), 1.52 (3H, s), 1.16 (3H, d, J=7.2 Hz), 1.14 (3H, d, J=7.2 Hz); FAB-MS m/z 526 [M+H]⁺.

Compound 19:

¹H-NMR (CDCl₃, 300 MHz) δ(ppm): 7.00 (2H, d, J=8.6 Hz), 6.79 (2H, d, J=8.6 Hz), 6.57 (1H, d, J=11.6 Hz), 6.13 (1H, m), 6.09 (1H, brs), 5.59 (1H, d, J=11.6 Hz), 5.31 (1H, m), 3.81 (2H, m), 3.72 (3H, s), 3.36 (1H, brt, J=6.8 Hz), 2.87 (1H, m), 2.76-2.60 (4H, m), 2.09 (1H, brd, J=10.3 Hz), 1.61 (3H, s), 1.44 (6H, s), 1.10 (3H, d, J=6.8 Hz); FAB-MS m/z 526 [M+H]⁺.

### EXAMPLE 12

### Compound 20

A loopful of cells from an agar slant culture of the fungi MPC1006 strain was inoculate into each of the two flasks (300 mL) containing 50 mL seed medium (mashed potato 3%, glucose 10%, yeast extracts 0.5%, pH 6.5) and cultured at 28° C. for 5 days on a rotary shaker to obtain the seed cultures. Each of the 40 flasks (300 mL) containing 50 mL production medium (glucose 2%, mashed potato 2%, peptone 0.5%, KH₂PO₄ 0.5%, Mg₃(PO₄)₂.8H₂O 0.05%, pH 6.0) was inoculated with 2.5 mL of the seed culture and cultured at 25° C. for 5 days on the rotary shakers. After the completion of culturing, the culture (2 L) was separated into fungi cells and a supernatant by suction filtration.

The culture supernatant was charged to a column of Diaion HP-20 (30 mL) which had been equilibrated with 20% methanol. The column was washed stepwise with 40, 50, 60% methanol and then eluted with 90 and 100% methanol. The eluate (150 mL) was concentrated and extracted with ethyl acetate. The extract was purified by a high speed liquid chromatography (Develosil HG-5 20×250 mm; eluted stepwise with 40-100% acetonitryl solution) to obtain 2.2 mg of Compound 20.

Compound 20:

H-NMR (CDCl₃, 400 MHz) δ(ppm): 7.15 (1H, dd, J=2.2, 15.4 Hz), 6.25 (1H, brs), 6.19 (1H, brd, J=11.0 Hz), 6.03 (1H, dd, J=2.2, 15.4 Hz), 5.30 (1H, m), 4.55 (1H, m), 3.88 (1H, dd, J=2.4, 7.6 Hz), 3.82 (1H, brd, J=11.0 Hz), 3.17 (1H, dt, J=10.0, 3.4 Hz), 3.09 (1H, m), 2.93 (1H, dd, J=3.2, 4.9 Hz), 2.39-2.34 (1H, m), 2.20-2.08 (2H, m), 1.73 (3H, s), 1.61 (1H, m), 1.50-1.46 (1H, m), 1.43-1.34 (1H, m), 1.39 (3H, s), 1.33-1.25 (1H, m), 1.21 (3H, d, J=7.3 Hz), 0.931 (3H, d, J=6.6 Hz), 0.926 (3H, d, J=6.6 Hz); ¹³C-NMR (CDCl₃, 100 MHz) δ(ppm): 173.6, 167.5, 150.4, 140.2, 139.5, 124.2, 122.8, 120.1, 88.3, 78.1, 73.9, 52.1, 51.7, 48.5, 39.39, 39.36, 34.2, 28.0, 25.2, 23.6, 21.4, 19.7, 15.2, 13.8; FAB-MS m/z 418 [M+H]⁺.

### EXAMPLE 13

### Compound 21

A loopful of cells from an agar slant culture of the fungi MPC1009 strain was inoculate into each of the 4 flasks (300 mL) containing 50 mL of primary seed medium (mashed potato 3%, glucose 10%, yeast extracts 0.5%, pH 6.5) and cultured at 28° C. for 4 days on a rotary shaker to obtain a primary seed culture. The secondary seed medium (glucose 2%, mashed potato 2%, peptone 0.5%, KH₂PO₄ 0.5%, Mg₃(PO₄)₂ .8H₂O 0.05%, pH 6.0) 2.5 L was inoculated with 75 mL of the primary seed culture and cultured at 25° C. for 24 hours in a 5 L jar fermenter with aeration. Each of the two 30 L jar fermenter was loaded with 15 L of the production medium (glucose 2%, mashed potato 2%, peptone 0.5%, KH₂PO₄ 0.5%, Mg₃(PO₄)₂.8H₂O 0.05%, pH 6.0), inoculated with 450 mL of the secondary seed culture and cultured at 25° C. for 5 days with aeration. After the completion of culturing, the culture (30 L) was filtered by suction to separate a culture into fungi cells and a supernatant. The fungi cells were extracted by stirring in 20 mL methanol. The extract was charged to a column of Diaion HP-20 (1.5 L) which had been equilibrated with 30% methanol, washed with 50% methanol and then eluted with 100% methanol. The eluate was concentrated, extracted with ethyl acetate and dried to obtain 23.7 g of the cell extract. The cell extract was subjected to silica gel column chromatography (a mixed solvent of chloroform and methanol), and the fractions containing Compound 21 were collected, concentrated and dried. The resulting residue was subjected to partition with 90% methanol and n-hexane to remove fatty acids. The extract (5.44 g) in 90% methanol was charged to a silica gel column and eluted with n-hexane and ethyl acetate mixture solvent. The fractions containing Compound 21 was collected and concentrated. The resulting residue (1.24 mg) was dissolved in a small amount of ether and Compound 21 (343 mg) in powder form was obtained by adding n-hexane.

Compound 21:

H-NMR (CDCl₃, 500 MHz) δ(ppm): 7.33 (1H, dd, J=2.5, 15.4 Hz), 6.94 (1H, brs), 6.22 (1H, d, J=10.8 Hz), 5.91 (1H, dd, J=2.1, 15.4 Hz), 5.29 (0.1H, brs), 4.51 (1H, m), 3.80 (1H, d, J=10.8 Hz), 3.14 (1H, ddd, J=3.0, 3.0, 10.3 Hz), 3.08 (1H, m), 2.89 (1H, dd, J=3.3, 4.8 Hz), 2.23 (1H, dd, J=9.6, 13.6 Hz), 2.11 (1H, m), 2.01 (1H, m), 1.88 (1H, brs), 1.75 (1H, m), 1.73 (3H, s), 1.68 (1H, m), 1.63 (1H, m), 1.53 (1H, m), 1.50 (1H, m), 1.44 (3H, s), 1.28 (1H, m), 1.20 (3H, d, J=7.3 Hz), 0.92 (3H, d, J=6.8 Hz), 0.91 (3H, d, J=6.8 Hz); ¹³C-NMR (CDCl₃, 125 MHz) δ(ppm): 173.7, 167.7, 156.5, 140.1, 139.4, 124.4, 123.0, 118.8, 88.4, 69.2, 52.2, 51.9, 48.6, 42.2, 39.5, 38.1, 34.2, 25.0, 23.8, 21.3, 19.8, 17.8, 16.2, 13.9; FAB-MS m/z 402 [M+H]⁺.

### EXAMPLE 14

### Compound 16

A loopful of cells from an agar slant culture of the fungi MPC1005 strain was inoculated into each of the 4 flasks (300 mL) containing 50 mL seed medium (mashed potato 3%, glucose 10%, yeast extracts 0.5%, pH 6.5) and cultured at 28° C. for 5 days by using a rotary shaker to obtain a seed cultures. Each of the 50 flasks (300 mL) containing 50 mL production medium (sucrose 3%, soluble starch 2%, dried yeast 0.5%, malt extracts 1%, corn steep liquor (CSL) 0.5%, vegetable juice 20%, CaCO₃ 0.5%, pH 6.0) was inoculated with 2.5 mL of the seed culture and cultured at 25° C. for 5 days by using the rotary shakers.

After the completion of culturing, a culture (2.5 L) was filtered by sunction to separate the culture into fungi cells and a supernatant. The fungi cells were mixed with 6 L of in ethanol, stirred and extracted. The methanol extract was concentrated to about 3 L under reduced pressure. The culture supernatant and the concentrated cell extract were combined and charged to a column of Diaion HP-20 (150 mL) which had been equilibrated with 50% methanol. The column was washed with 50% methanol and then eluted with 100% methanol. The eluate was concentrated to about 200 mL under reduced pressure, extracted with ethyl acetate (250 mL×2). The organic layer washed with a saturated aqueous sodium chloride solution, dried with anhydrous sodium sulfate, concentrated and dried. The resulting residue (1.58 g) was dissolved into a small quantity of methanol and charged to a Sephadex LH-20 column (internal diameter 3.0 cm, height 20 cm) which had been equilibrated with a mixed solvent of chloroform and methanol (1:1), and eluted with a mixed solvent of chloroform and methanol. The eluate was fractionated into 20 mL fractions and the fractions containing Compound 16 were collected and concentrated to dryness under reduced pressure. The dried material was subjected to silica gel column chromatography (50 ml, a mixed solvent of methanol and chloroform) and Compound 16 (18 mg) was obtained.

Compound 16:

¹H-NMR (CDCl₃, 500 MHz) δ(ppm): 7.05 (2H, d, J=8.5 Hz), 6.81 (2H, d, J=8.5 Hz), 6.66 (1H, brs), 6.39 (1H, m), 5.79 (1H, ddd, J=1.7, 10.0, 15.1 Hz), 5.44 (1H, ddd, J=3.9, 11.2, 15.1 Hz), 3.62 (1H, t, J=5.9 Hz), 3.33 (1H, m), 3.32 (1H, m), 3.02 (1H, dd, J=6.8, 12.1 Hz), 2.89 (1H, dd, J=5.4, 10.0 Hz), 2.75 (2H, m), 2.67 (1H, dd, J=2.1, 5.9 Hz), 2.59 (1H, d, J=5.4 Hz), 2.25 (1H, m), 2.24 (1H, m), 2.03 (1H, dt, J=13.8, 11.6 Hz), 1.88 (3H, d, J=0.7 Hz), 1.17 (3H, s), 1.15 (3H, d, J=6.6 Hz), 1.11 (3H, d, J=7.3 Hz); ¹³C-NMR (CDCl₃, 125 MHz) δ(ppm): 206.2, 171.9, 169.1, 155.1, 143.1, 135.1, 132.7, 130.8, 130.6, 128.7, 126.1, 115.8, 84.9, 60.4, 57.2, 54.4, 49.5, 47.0, 43.9, 39.9, 39.8, 36.8, 36.1, 19.7, 17.4, 13.0, 12.8; FAB-MS m/z 480 [M+H]⁺.

### REFERENCE EXAMPLE 1

### Compound 1 (UCS15A/Luminacin C2/SI-4228A)

Compound 1 was obtained by isolating and purifying from the fermentation broth of an actinomycete strain Streptomiyces sp., which is capable of producing Compound 1, according to the methods described in The Journal of Antibiotics, Vol. 53, p. 579 (2000) and Japanese Published Unexamined Patent Application No.116686/83.

Compound 1 (UCS15A/Luminacin C2/SI-4228A):

¹H-NMR (CDCl₃, 300 MHz) δ(ppm): 14.16 (1H, s), 12.93 (1H, s), 10.35 (1H, s), 8.01 (1H, s), 4.85 (1H, d, J=3.2 Hz), 4.36 (1H, dd, J=8.1, 9.6 Hz), 4.27 (1H, d, J=7.0 Hz), 4.11 (1H, ddd, J=4.6, 11.4, 11.4 Hz), 3.66 (1H, m), 3.53 (1H, d, J=3.2 Hz), 3.19 (1H, t, J=6.2 Hz), 3.14 (3H, s), 1.85-1.98 (2H, m), 1.69-1.78 (2H, m), 1.40-1.58 (3H, m), 1.08-1.19 (2H, m), 0.98 (3H, t, J=7.5 Hz), 0.91 (3H, d, J=6.6 Hz), 0.79 (3H, t, J=7.3 Hz), 0.77 (3H, d, J=6.8 Hz); FAB-MS m/z 479 [M−H]⁻.

### REFERENCE EXAMPLE 2

### Compound 5

Compound 5 can be obtained in a manner similar to that described in Japanese Published Unexamined Patent Application No.294619/87, but also can be obtained by the following method.

Compound 1 (11 mg; 0.023 mmol) obtained in Reference Example 1 was dissolved in a mixed solvent of THF (1 mL), water (0.5 mL) and a drop of methanol, methylhydrazine sulfate (30 mg; 0.21 mmol) was added thereto. The mixture was stirred for 4 hours at room temperature. The reaction mixture was diluted with ethyl acetate (10 mL) and extracted. The organic layer was washed with water and 1 mol/L hydrochloric acid sequentially, and then dried over anhydrous magnesium sulfate and concentrated. The resulting residue was recrystallized from a mixed solvent of IPE and n-hexane to obtain 5.9 mg of Compound 5 (yield 50%). Compound 5:

¹H-NMR (CDCl₃) δ(ppm): 0.84 (3H, t, J=7.2 Hz), 0.86 (3H, d, J=6.8 Hz), 0.96 (3H, d, J=6.6 Hz), 1.06 (3H, t, J=7.5 Hz), 1.21 (2H, m), 1.42-1.62 (3H, m), 1.82 (2H, m), 2.01 (2H, m), 3.00 (3H, s), 3.22 (3H, s), 3.26 (1H, t, J=6.2 Hz), 3.70 (1H, m), 4.16 (1H, m), 4.35 (1H, d, J=6.6 Hz), 4.40 (1H, m), 4.98 (1H, s), 6.72 (1H, s), 7.74 (1H, s), 8.21 (1H, s), 13.00 (1H, brs), 13.71 (1H, s); FAB-MS m/z 477 [M—CH₃NH₂]⁺, 507 [M−H]⁻.

Compounds 7, 8 and 9 were obtained in a manner similar to that described in J. Am. Chem. Soc., Vol. 122, p. 3071 (2000)

### REFERENCE EXAMPLE 3

### Compound 7

Resorcinol (600 mg; 5.45 mmol) and sorbic acid (600 mg; 5.36 mmol) were dissolved in boron trifluoride diethyl etherate complex (10 mL) and stirred for 15 minutes at 120° C. While the reaction mixture was cooled under ice-cooling, a small amount of water was added thereto, and extracted with ethyl acetate (50 ml×2). The solvent was distilled away from the organic layer under reduced pressure, and the resulting residue was dissolved in a mixed solvent of THF (50 mL) and water (50 mL) and heated for 30 minutes under reflux. After the reaction mixture was concentrated and THF was removed. The resulting residue was extracted with ethyl acetate (50 ml×2). After the organic layer was dried over anhydrous magnesium sulfate, and concentrated. The resulting residue was purified by silica gel column chromatography (75 g; n-hexane/ethyl acetate=3/1) to obtain 566 mg of Compound 7 (yield 60%).

Compound 7:

¹H-NMR (CDCl₃) δ(ppm): 1.92 (3H, d, J=5.1 Hz), 5.57 (1H, brs), 6.32-6.40 (4H, m), 6.91 (1H, d, J=14.7 Hz), 7.48 (1H, dd, J=9.9, 14.7 Hz), 7.71 (1H, d, J=9.2 Hz), 13.41 (1H, s); FAB-MS m/z 205 [M+H]⁺.

### REFERENCE EXAMPLE 4

### Compound 8

Compound 8 (736 mg, yield 84%) was obtained from 2-methylresolcinol (500 mg; 4.03 mmol) and sorbic acid (450 mg; 4.01 mmole) in a manner similar to that in Reference Example 3.

Compound 8:

¹H-NMR (CDCl₃) δ(ppm): 1.90 (3H, d, J=5.5 Hz), 2.14 (3H, s), 6.00 (1H, brs), 6.23-6.35 (2H, m), 6.39 (1H, d, J=9.0 Hz), 6.92 (1H, d, J=15.0 Hz), 7.46 (1H, dd, J=9.9, 15.0 Hz), 7.58 (1H, d, J=9.0 Hz), 13.73 (1H, s); FAB-MS m/z 219 [M+H]⁺.

### REFERENCE EXAMPLE 5

### Compound 9

Compound 9 (971 mg, yield 53%) was obtained from 2,4-dimethylresolcinol (1.08 g; 7.83 mmol) and sorbic acid (950 mg; 8.47 mmol) in a manner similar to that in Reference Example 3.

Compound 9:

¹H-NMR (CDCl₃) δ1.91 (3H, d, J=5.7 Hz), 2.15 (3H, s), 2.22 (3H, s), 5.32 (1H, s), 6.24-6.40 (2H, m), 6.95 (1H, d, J=14.9 Hz), 7.45 (1H, s), 7.46 (1H, dd, J=10.1, 14.9 Hz), 13.60 (1H, s); FAB-MS m/z 233 [M+H]⁺.

### REFERENCE EXAMPLE 6

### Compound 10

Compound 10 can be obtained in a manner similar to that described in Tetrahedron, VOL. 31, p. 1593 (1975), but it was also possible to be obtained by methylation of Compound 7.

Compound 7 (424 mg; 2.08 mmol) obtained in Reference Example 3 was dissolved in 30 mL of acetone, calcium carbonate (650 mg; 4.70 mmol) and methyl iodide (0.260 mL; 4.18 mmol) were added thereto. The mixture was heated for 1 hour under reflux. The reaction mixture was allowed to cool, and then was diluted with ethyl acetate (50 mL) and extracted after addition of 1 mol/L hydrochloric acid. The organic layer was washed with saturated sodium chloride, dried over anhydrous magnesium sulfate and concentrated. The resulting residue was purified by silica gel column chromatography (30 mL; n-hexane/ethyl acetate=4/1) to obtain 113 mg of Compound 10 (yield 25%).

Compound 10:

¹H-NMR (CDCl₃) δ(ppm): 1.91 (3H, d, J=5.0 Hz), 3.85 (3H, s), 6.26-6.40 (2H, m), 6.43-6.47 (2H, m), 6.92 (1H, d, J=14.9 Hz), 7.48 (1H, dd, J=9.9, 14.9 Hz), 7.71 (1H, d, J=9.5 Hz), 13.50 (1H, s); FAB-MS m/z 219 [M+H]⁺

### REFERENCE EXAMPLE 7

### Compound 11

Compound 7 (137 mg; 0.972 mmol) was dissolved in 25 mL acetone, calcium carbonate (1.50 g; 10.85 mmol) and methyl iodide (1.50 ml; 24.1 mmol) were added thereto. The mixture was heated for 3 hours under reflux. The reaction mixture was treated in a manner similar to that in Reference Example 6 to obtain 122 mg of Compound 11 (yield 78%).

Compound 11:

¹H-NMR (CDCl₃) δ(ppm): 1.85 (3H, d, J=6.2 Hz), 3.83 (3H, s), 3.84 (3H, s), 6.12-6.32 (2H, m), 6.46 (1H, d, J=2.2 Hz), 6.51 (1H, dd, J=2.2, 8.6 Hz), 6.81 (1H, d, J=15.2 Hz), 7.25 (1H, dd, J=11.0, 15.0 Hz), 7.66 (1H, d, J=8.6 Hz); FAB-MS m/z 233 [M+H]⁺.

### REFERENCE EXAMPLE 8

### Compound 13 (Cytochalasin E)

Compound 13 was obtained by isolating and purifying from the culture medium of fungi which is capable of producing Compound 13 in a manner similar to that described in J. Chem. Soc. Perkin Trans. 1, p. 541 (1982), Agric. Biol. Chem., Vol. 53, p. 1699 (1989), etc.

Compound 13: (Cytochalasin E):

¹H-NMR (CDCl₃, 300 MHz) δ(ppm): 7.27-7.37 (3H, m), 7.15 (2H, d, J=7.0 Hz), 6.53 (1H, d, J=12.5 Hz), 6.00 (1H, brs), 5.89 (1H, dd, J=8.1, 15.0 Hz), 5.63 (1H, d, J=12.5 Hz), 5.23 (1H, m), 3.71 (1H, m), 3.02 (1H, m), 2.94 (1H, m), 2.89 (1H, dd, J=4.2, 13.9 Hz), 2.50-2.70 (4H, m), 2.31 (1H, m), 2.15 (1H, brd, J=15.0 Hz), 1.50 (3H, s), 1.25 (3H, s), 1.16 (3H, d, J=6.1 Hz), 1.12 (3H, d, J=6.8 Hz); FAB-MS m/z 496 [M+H]⁺

### REFERENCE EXAMPLE 9

### Compound 12 (Mer-WF1726)

Compound 12 can be obtained in a manner similar to that described in Japanese Published Unexamined Patent Application No. 114776/98.

Compound 12 (Mer-WF1726):

¹H-NMR (CDCl₃, 500 MHz) δ (ppm): 7.06 (2H, d, J=8.5 Hz), 6.86 (2H, d, J=8.5 Hz), 6.51 (1H, d, J=11.6 Hz), 6.16 (1H, brs), 5.90 (1H, dd, J=9.3, 14.9 Hz), 5.61 (1H, d, J=11.6 Hz), 5.23 (1H, ddd, J=3.8, 10.9, 14.9 Hz), 3.79 (3H, s), 3.68 (1H, m), 3.00 (1H, dd. J=2.6, 5.1 Hz), 2.94 (1H, m), 2.82 (1H, dd, J=4.5, 13.7 Hz), 2.69-2.60 (4H, m), 2.29 (1H, dq, J=12.5, 7.3 Hz), 2.15 (1H, brd, J=13.8 Hz), 1.49 (3H, s), 1.25 (3H, s), 1.16 (3H, d, J=6.8 Hz), 1.10 (3H, d, J=7.3 Hz); ¹³C-NMR (CDCl₃, 125 MHz) δ(ppm): 211.8, 170.0, 158.9, 149.4, 142.2, 131.6, 130.6, 128.5, 127.9, 120.5, 114.4, 87.1, 77.3, 60.7, 57.3, 55.3, 53.8, 48.0, 45.9, 44.1, 40.9, 39.1, 35.9, 24.4, 20.1, 19.7, 13.3; FAB-MS m/z 526 [M+H]⁺.

### REFERENCE EXAMPLE 10

### Compound 26 (Aspochalasin C), Compound 27 (Aspochlasin D) and Compound 28 (Aspochalasin E)

Compound 26 (Aspochalasin C), Compound 27 (Aspochlasin D) and Compound 28 (Aspochalasin E) were obtained by culturing the fungi MPC1009 strain, isolating and purifying from the culture medium of that by the methods described in the Example 13.

Said compounds can also be obtained by culturing the fungi which can produce said Compounds, isolating and purifying from the culture medium of that in a manner similar to that described in Helv. Achim. Acta, Vol. 62, p. 1501 (1979), J. Antibiot., Vol. 46, p. 679 (1993), etc. Compound 26 (Aspochalasin C):

¹H-NMR (CDCl₃, 400 MHz) δ(ppm): 7.32 (1H, dd, J=0.7, 16.3 Hz), 6.26 (1H, dd, J=8.3, 16.3 Hz), 6.07 (1H, brs), 5.99 (1H, brd, J=11.2 Hz), 5.41 (1H, m), 3.95 (1H, t, J=8.3 Hz), 3.49 (1H, m), 3.10 (1H, m), 3.02 (1H, dd, J=3.7, 5.4 Hz), 2.82 (1H, brd, J=11.0 Hz), 2.46 (2H, m), 2.02 (1H, m), 1.93 (1H, m), 1.85 (1H, ddd, J=2.7, 13.2, 13.2 Hz), 1.77 (3H, d, J=1.5 Hz), 1.55 (1H, m), 1.41 (3H, d, J=1.5 Hz), 1.24 (2H, m), 1.23 (3H, d, J=7.3 Hz), 0.91 (3H, d, J=6.6 Hz), 0.90 (3H, d, J=6.6 Hz); ¹³C-NMR (CDCl₃, 100 MHz) δ(ppm): 198.2, 174.4, 140.8, 138.3 (2 C), 130.3, 125.8, 125.5, 77.3, 75.1, 68.0, 51.3, 49.7, 48.4, 43.5, 36.4, 36.3, 35.2, 25.2, 23.7, 21.4, 20.0, 19.9, 13.7; FAB-MS m/z 402 [M+H]⁺.

Compound 27 (Aspochlasin D):

¹H-NMR (CDCl₃, 300 MHz) δ(ppm): 7.14 (1H, d, J=16.7 Hz), 6.14 (1H, dd, J=5.0, 16.5 Hz), 6.33 (1H, brs), 5.94 (1H, d, J=10.8 Hz), 5.42 (1H, brs), 4.58 (1H, brs), 3.78 (1H, brs), 3.15 (1H, m), 3.00 (1H, dd, J=2.8, 5.7 Hz), 2.90 (1H, d, J=10.8 Hz), 2.48 (1H, m), 2.16 (2H, m), 2.07 (1H, m), 1.75 (3H, s), 1.50 (2H, m), 1.30 (3H, s), 1.22 (3H, d, J=7.3 Hz), 1.21 (1H, m), 0.90 (3H, d, J=6.4 Hz), 0.88 (3H, d, J=6.4 Hz); ¹³C-NMR (CDCl₃, 75 MHz) δ(ppm): 197.6, 174.9, 141.5, 140.4, 137.4, 129.6, 125.7, 124.2, 79.2, 75.6, 68.0, 51.0, 49.6, 48.3, 43.6, 39.5, 35.1, 29.3, 25.1, 23.6, 21.5, 19.9, 15.6, 13.5; FAB-MS m/z 402 [M+H]⁺.

Compound 28 (Aspochalasin E):

¹H-NMR (DMSO-d₆, 500 MHz) δ(ppm): 8.10 (1H, s), 6.02 (1H, d, J=10.9 Hz), 5.33 (1H, brs), 4.41 (1H, d, J=5.9 Hz), 4.37 (1H, d, J=4.0 Hz), 4.35 (1H, brs), 3.90 (1H, d, J=17.6 Hz), 3.60 (1H, brs), 3.24 (1H, brs), 3.17 (1H, brs), 3.07 (2H, m), 2.48-2.40 (2H, m), 2.00-1.94 (2H, m), 1.76 (1H, dd, J=5.2, 17.6 Hz), 1.70 (3H, s), 1.60-1.44 (2H, m), 1.38 (3H, d, J=1.1 Hz), 1.31 (1H, m), 1.16 (3H, d, J=6.9 Hz), 1.09-0.98 (2H, m), 0.84 (3H, d, J=6.6 Hz), 0.83 (3H, d, J=6.5 Hz); FAB-MS m/z 420 [M+H]⁺.

### REFERENCE EXAMPLE 11

### Compound 22 and Compound 25 (Aspochalasin B)

Compound 27 (31.7 mg; 0.0791 mmol) obtained in Reference Example 10 was dissolved in dichloromethane (5 mL), manganese dioxide (250 mg; 2.44 mmol) was added thereto. The mixture was stirred for 2 hours at room temperature. The reaction mixture was filtered through Celite. The filtrate was concentrated and the resulting residue was purified by preparative silica gel thin-layer chromatography (a mixed solvent of chloroform and methanol) to obtain Compounds 25 (13.6 mg; yield 43%) and 22 (5.1 mg; yield 16%).

Compound 25 (Aspochalasin B) can also be obtained by culturing the fungi which can produce Compound 25, isolating and purifying from the culture medium of that in a manner similar to that described in Helv. Chim. Acta, Vol. 62, p. 1501 (1979). Compound 25 (Aspochalasin B):

¹H-NMR (CDCl₃, 300 MHz) δ(ppm): 8.28 (1H, d, J=16.5 Hz), 6.52 (1H, d, J=16.5 Hz), 6.33 (1H, d, J=11.2 Hz), 5.99 (1H, brs), 5.44 (1H, brs), 4.82 (1H, brd, J=6.1 Hz), 3.59 (1H, brs), 3.19 (1H, t, J=4.6 Hz), 3.11 (1H, m), 2.74 (1H, brd, J=11.4 Hz), 2.62 (1H, m), 2.38 (2H, m), 1.86 (1H, m), 1.79 (3H, brs), 1.57 (3H, m), 1.24 (3H, d, J=7.0 Hz), 1.22 (3H, s), 0.92 (3H, d, J=5.9 Hz), 0.90 (3H, d, J=6.2 Hz); ¹³C-NMR (CDCl₃, 75 MHz) δ(ppm): 205.1, 195.5, 173.2, 141.6, 138.8, 136.4, 126.5 (2 C), 124.7, 74.7, 68.9, 51.7, 48.2, 47.5, 41.6, 39.7, 34.8, 32.4, 25.2, 23.7, 21.1, 20.2, 15.5, 13.8; FAB-MS m/z 400 [M+H]⁺.

Compound 22:

H-NMR (CDCl₃, 300 MHz) δ(ppm): 9.77 (1H, brs), 9.75 (1H, d, J=7.7 Hz), 7.76 (1H, d, J=15.8 Hz), 6.78 (1H, dd, J=7.7, 15.8 Hz), 6.12 (1H, brs), 5.76 (1H, d, J=10.3 Hz), 5.34 (1H, brs), 3.23 (1H, d, J=9.9 Hz), 3.16 (1H, m), 2.74 (1H, t, J=4.8 Hz), 2.54 (3H, m), 2.33 (2H, m), 1.76 (3H, brs), 1.54 (1H, m), 1.50 (3H, s), 1.34 (1H, m), 1.21 (3H, d, J=7.3 Hz), 0.92 (6H, d, J=6.6 Hz); FAB-MS m/z 400 [M+H]⁺.

### REFERENCE EXAMPLE 12

### Compound 24 (Aspochalasin A)

Compound 25 (28.9 mg; 0.0724 mmol) obtained in Reference Example 11 was dissolved in pyridine (0.5 mL), triethylamine (0.5 mL) was added thereto. The mixture was stirred for 19 hours at room temperature. The reaction mixture was poured in to water, and extracted with chloroform (20 mL). The organic layer was washed with 0.5 mol/L hydrochloric acid. The resulting extract was purified by preparative silica gel thin-layer chromatography (chloroform-methanol) to obtain Compound 24 (21.2 mg; yield 73%).

Compound 24 (Aspochalasin A) can also be obtained by culturing the fungi which can produce Compound 24, isolating and purifying from the culture medium of that in a manner similar to that described in Helv. Chim. Acta, Vol. 62, p. 1501 (1979).

Compound 24

¹H-NMR (CDCl₃, 300 MHz) δ(ppm): 6.73 (1H, brs), 6.22 (1H, d, J=11.0 Hz), 5.32 (1H, brs), 3.94 (1H, dd, J=12.8, 19.4 Hz), 3.67 (1H, m), 3.14-3.07 (2H, m), 2.94 (1H, brd, J=9.5 Hz), 2.79 (1H, brt, J=12.8 Hz), 2.55-2.40 (3H, m), 2.38-2.02 (2H, m), 1.99 (1H, m), 1.72 (3H, s), 1.56 (1H, m), 1.48 (3H, s), 1.19 (3H, J=6.6 Hz), 1.16 (2H, m), 0.94 (6H, d, J=6.6 Hz); ¹³C-NMR (CDCl₃, 75 MHz) δ(ppm): 208.7, 202.6, 197.6, 175.5, 140.0, 135.7, 125.1, 124.8, 66.3, 52.1, 50.7, 48.7, 43.4, 38.8, 36.6, 35.3, 31.4, 31.2, 25.0, 23.5, 21.4, 19.9, 14.8, 13.4; FAB-MS m/z 400 [M+H]⁺.

### REFERENCE EXAMPLE 13

### Compound 23

Compound 25 (216 mg; 0.541 mmol) obtained in Reference Example 11 was dissolved in ethyl acetate (30 mL), 10% palladium-carbon (150 mg) was added thereto. The mixture was stirred for 4 hours at room temperature under hydrogen atmosphere. After removing the catalyst by filtration, the reaction mixture was purified by preparative silica gel thin-layer chromatography to obtain 92.3 mg of Compound 23 (yield 43%).

Compound 23:

¹H-NMR (CDCl₃, 300 MHz) δ(ppm): 7.01 (1H, brs), 6.00 (1H, d, J=10.6 Hz), 4.17 (1H, brs), 3.40 (1H, m), 2.89-2.76 (3H, m), 2.42-1.76 (7H, m), 1.64-1.44 (2H, m), 1.32 (3H, d, J=1.1 Hz), 1.25-1.03 (2H, m), 0.92-0.85 (15H, m); FAB-MS m/z 402 [M+H]⁺.

### INDUSTRIAL APPLICABILITY

The present invention provides SH3 domain binding inhibitors comprising a non-peptide compound or a pharmaceutically acceptable salt thereof as an active ingredient. The present invention also provides compounds or pharmaceutically acceptable salts thereof which are useful as an SH3 domain binding inhibitor.

1. A method for inhibiting SH3 domain binding, which comprises administering an effective amount of a non-peptide compound exhibiting SH3 domain binding inhibitory activity or a pharmaceutically acceptable salt thereof.

2. The method for inhibiting SH3 domain binding according to claim 1, wherein said non-peptide compound is a low molecular weight compound with molecular weight of less than 750.

3. The method for inhibiting SH3 domain binding inhibitor according to claim 1, wherein said non-peptide compound exhibiting SH3 domain binding inhibitory activity is a compound represented by the general formula (I): 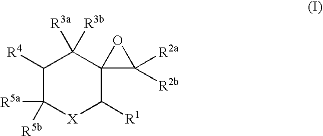
(wherein R¹, R^{3a}, R^{3b} and R⁴ may be the same or different and represent a hydrogen atom, hydroxy, carboxy, substituted or unsubstituted lower alkyl, substituted or unsubstituted lower alkoxy, or substituted or unsubstituted lower alkanoyloxy; or R^{3a} and R^{3b} are combined to represent an oxygen atom; R^{2a} and R^{2b} may be the same or different and represent a hydrogen atom, substituted or unsubstituted lower alkyl, or substituted or unsubstituted alkenyl; R^{5a} and R^{5b} may be the same or different and represent a hydrogen atom, hydroxy, carboxy, substituted or unsubstituted lower alkyl, substituted or unsubstituted lower alkoxy, substituted or unsubstituted lower alkenyl, substituted or unsubstituted lower alkanoyloxy, substituted or unsubstituted lower alkenoyloxy, or substituted or unsubstituted lower alkanoylaminocarbonyloxy; or R^{5a} and R^{5b} are combined to represent an oxygen atom; and X represents an oxygen atom or —CH₂—).

4. The method for inhibiting SH3 domain binding according to claim 1, wherein said non-peptide compound exhibiting SH3 domain binding inhibitory activity is a compound represented by the general formula (II): 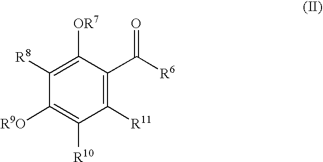
(wherein R⁶ represents a hydrogen atom, substituted or unsubstituted lower alkyl, or substituted or unsubstituted lower alkenyl; R⁷ and R⁹ may be the same or different and represent a hydrogen atom, formyl, substituted or unsubstituted lower alkyl, substituted or unsubstituted lower alkanoyl, or substituted or unsubstituted lower aralkyl; and R⁸, R¹⁰ and R¹¹ may be the same or different and represent a hydrogen atom, halogen, hydroxy, carboxy, substituted or unsubstituted lower alkyl, substituted or unsubstituted lower alkoxy, substituted or unsubstituted lower alkanoyl, formyl, cyano, nitro, amino, mono- or di-lower alkylamino, substituted or unsubstituted lower alkanoylamino, or substituted or unsubstituted alkoxycarbonyl).

5. The method for inhibiting SH3 domain binding according to claim 1, wherein said non-peptide compound exhibiting SH3 domain binding inhibitory activity is a cytochalasin.

6. The method for inhibiting SH3 domain binding according to claim 1, wherein said non-peptide compound exhibiting SH3 domain binding inhibitory activity is a compound represented by the general formula (IIIa): 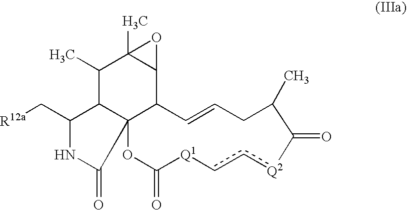
(wherein R^{12a} represents substituted or unsubstituted lower alkyl, substituted or unsubstituted aryl, or substituted or unsubstituted heteroaryl; Q¹ represents a single bond or an oxygen atom; 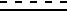
represents a single bond or a double bond; and when the 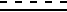
between Q² and a carbon atom adjacent thereto represents a double bond, =Q²- represents ═C(CH₃)—, and when the 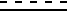
between Q² and a carbon atom adjacent thereto represents a single bond, -Q²- represents —C(OH)(CH₃)).

7. The method for inhibiting SH3 domain binding according to claim 1, wherein said non-peptide compound exhibiting SH3 domain binding inhibitory activity is a compound represented by the general formula (IIIb): 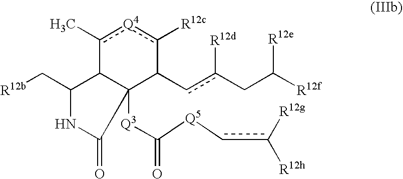
(wherein R^{12b} has the same meaning as the above-described R^{12a}; Q³ and Q⁵ may be the same or different and represent a single bond or an oxygen atom; 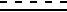
represents a single bond or a double bond; 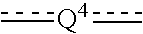
represents ═C(CH₃)—, —C(═CH₂)—, —CH(CH₃)— or —C(CH₃)═; R^{12c} and R^{12h} may be the same or different and represent a hydrogen atom or hydroxy; R^{12d} and R^{12e} may be the same or different and represent a hydrogen atom or methyl; and R^{12f} and R^{12g} represent formyl, or R^{12f} and R^{12g} are combined and 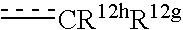
and —CHR^{12e}R^{12f} form 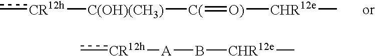
(wherein A and B may be the same or different and represent —CH(OH)—, —CH₂— or —C(═O)—)).

8. The method for inhibiting SH3 domain binding according to claim 3, wherein X represents an oxygen atom; R^{2a}, R^{3a}, R⁴ and R^{5b} are hydrogen atoms; R¹ and R^{3b} may be the same or different and represent hydroxy, carboxy, substituted or unsubstituted lower alkoxy, or substituted or unsubstituted lower alkanoyloxy; R^{2b} represents substituted or unsubstituted lower alkyl; and R^{5a} represents the general formula (IV): 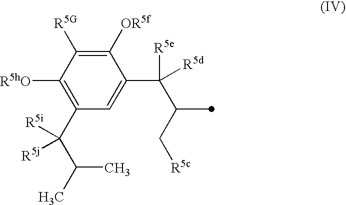
(wherein R^{5c} represents substituted or unsubstituted lower alkyl; R^{5d} and R^{5e} may be the same or different and represent a hydrogen atom, hydroxy, or substituted or unsubstituted lower alkoxy, or R^{5d} and R^{5e} are combined to represent an oxygen atom; R^{5f} and R^{5h} represent a hydrogen atom, formyl, substituted or unsubstituted lower alkyl, or substituted or unsubstituted lower alkanoyl; R^{5g} represents formyl, —CH═NQ (wherein Q represents substituted or unsubstituted lower alkoxy, substituted or unsubstituted aryloxy, substituted or unsubstituted aralkyloxy, substituted or unsubstituted lower alkylamino, substituted or unsubstituted arylamino, or substituted or unsubstituted arylsulfonylamino), substituted or unsubstituted lower alkyl, or substituted or unsubstituted lower alkanoyl; and R⁵ⁱ and R^{5j} may be the same or different and represent a hydrogen atom, hydroxy, or substituted or unsubstituted lower alkoxy, or R⁵ⁱ and R^{5j} are combined to represent an oxygen atom).

9. A compound represented by the general formula (Va): 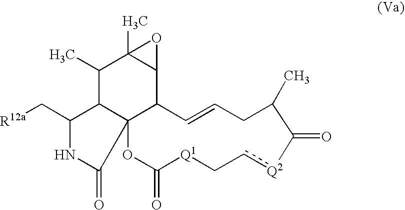
(wherein 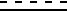
represents a single bond or a double bond; and R^{12a}, Q¹ and Q² have the same meanings as described above, respectively), or a pharmaceutically acceptable salt thereof.

10. A compound represented by the general formula (Vb): 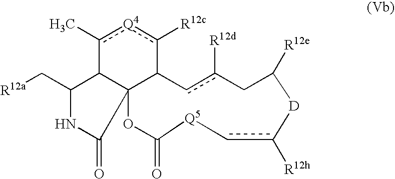
(wherein 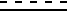
represents a single bond or a double bond; R^{12b}, R^{12c}, R^{12d}, R^{12e}, R^{12h}, Q⁵ and 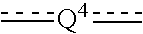
have the same meanings as described above, respectively; and 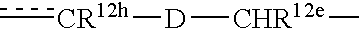
represents 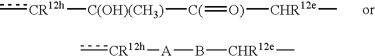
(wherein A and B have the same meanings as described above, respectively)), or a pharmaceutically acceptable salt thereof.

11. A compound represented by the general formula (VI) 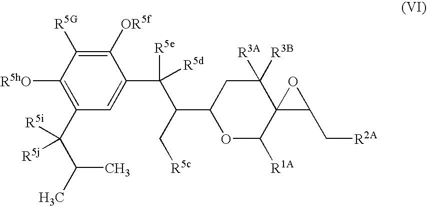
(wherein R^{1A}, R^{3A} and R^{3B} may be the same or different and represent a hydrogen atom, hydroxy, carboxy, substituted or unsubstituted lower alkoxy, or substituted or unsubstituted lower alkanoyloxy, or R^{3A} and R^{3B} are combined to represent an oxygen atom; R^{2A} represents substituted or unsubstituted lower alkyl; R^{5c}, R^{5d}, R^{5e}, R^{5f}, R^{5h}, R⁵ⁱ and R^{5j} have the same meanings as described above, respectively; and R^{5G} represents formyl, hydroxymethyl, substituted or unsubstituted lower alkoxymethyl, substituted or unsubstituted lower alkanoyloxymethyl, substituted or unsubstituted lower alkanoylmethyl, or —CH═NQ^{A} (wherein Q^{A} represents substituted or unsubstituted lower alkoxy, substituted or unsubstituted aryloxy, or substituted or unsubstituted aralkyloxy), with the proviso that when R^{5G} is formyl and one of R^{3A} and R^{3B} is a hydrogen atom, the other is not hydroxy], or a pharmaceutically acceptable salt thereof.

12. A pharmaceutical composition comprising the compound according to claim 9 or 10 or a pharmaceutically acceptable salt thereof as an active ingredient.

13. A pharmaceutical composition comprising the compound according to claim 11 or a pharmaceutically acceptable salt thereof as an active ingredient.

14. A method for inhibiting SH3 domain binding, which comprises administering an effective amount of the compound according to claim 9 or a pharmaceutically acceptable salt thereof.

15. A method for inhibiting SH3 domain binding, which comprises administering an effective amount of the compound according to claim 11 or a pharmaceutically acceptable salt thereof.

16. The method for inhibiting SH3 domain binding according to any one of claims 1-8 , wherein said SH3 domain binding is an interaction between a protein containing an SH3 domain and a protein containing a proline-rich sequence.

17. The method for inhibiting SH3 domain binding according to claim 16, wherein the protein containing an SH3 domain and/or the protein containing a proline-rich sequence are/is virus-derived protein(s).

18. The method for inhibiting SH3 domain binding according to claim 17, wherein said virus-derived protein is a retrovirus-derived protein, a hepatitis virus-derived protein or a herpes virus-derived protein.

19. The method for inhibiting SH3 domain binding according to claim 16, wherein said protein containing an SH3 domain is Src, Yes, Fgr, Hck, Lck, Abl, Fyn, Lyn, Blk, Yrk, Ras-GAP, PLCγ, P13K, Tec, Txk/Rlk, Tsk/Emt/Itk, Btk, Crk, Grb2, Nck, Vav, STAT, Cortactin, p40-phox, p67-phox, p47-phox, a TCR signaling molecule (TCRsm), or a β chain or a γ chain of IL-2R.

20. The method for inhibiting SH3 domain binding according to claim 16, wherein said protein containing a proline-rich sequence is HIV-1Nef, p22-phox, p47-phox, Sam68, Sos1, Dynamin, c-Cbl, ZO1, pX ORF I, LHDAg, NS5A, pORF3, ICP10, LMP2A, Tip or Tio.

21. The method for inhibiting SH3 domain binding according to claim 16, wherein said interaction between a protein containing an SH3 domain and a protein containing a proline-rich sequence is an interaction between Grb2 and Sos1, Fyn and Sam68, Src and Sam68, PLCγ and Sam68, Grb2 and Sam68, Lyn and HIV-1Nef, Hck and HIV-1Nef, TCRsm and HIV-1Nef, p47-phox and p22-phox, p67-phox and p47-phox, Lyn and Dynamin, Cortactin and ZO1, Lyn and c-Cb1, a β chain or a γ chain of IL-2R and pX ORF I, Grb2 and NS5A, Src and pORF3, Hck and pORF3, Fyn and pORF3, PI3K and pORF3, PLCγ and pORF3, Grb2 and pORF3, Grb2 and ICP10, Lyn and LMP2A, Lck and Tip, Lyn and Tio, Hck and Tio, Lck and Tio, Src and Tio, Fyn and Tio, or Yes and Tio.

22. The method for inhibiting SH3 domain binding according to claim 16, wherein said interaction between a protein containing an SH3 domain and a protein containing a proline-rich sequence is an interaction between Grb2 and Sos1, Fyn and Sam68, Src and Sam68, PLCγ and Sam68, Grb2 and Sam68, Lyn and HIV-1Nef, or Cortactin and ZO1.

23. A microorganism for producing the compound according to claim 9 or 10, selected from the group consisting of Xylariales filamentous fungus MPC1005 (Accession No.: FERM BP-7980), Aspergillus sp. MPC1006 (Accession No.: FERM BP-7899) and Aspergillus sp. MPC1009 (Accession No.: FERM BP-7900).

24-41. (canceled).

42. A method for treating and/or preventing a disease in which an SH3 domain binding is involved, which comprises administering an effective amount of the compound according to claim 9 or 10 or a pharmaceutically acceptable salt thereof.

43. A method for treating and/or preventing a disease in which an SH3 domain binding is involved, which comprises administering an effective amount of the compound according to claim 11 or a pharmaceutically acceptable salt thereof.

44. A method for treating a malignant tumor, which comprises administering an effective amount of the compound according to claim 9 or 10 or a pharmaceutically acceptable salt thereof.

45. A method for treating a malignant tumor, which comprises administering an effective amount of the compound according to claim 11 or a pharmaceutically acceptable salt thereof.

46. A method for treating and/or preventing an allergic disease, which comprises administering an effective amount of the compound according to claim 9 or 10 or a pharmaceutically acceptable salt thereof.

47. A method for treating and/or preventing an allergic disease, which comprises administering an effective amount of the compound according to claim 11 or a pharmaceutically acceptable salt thereof.

48. A method for treating a viral disease, which comprises administering an effective amount of the compound according to claim 9 or 10 or a pharmaceutically acceptable salt thereof.

49. A method for treating a viral disease, which comprises administering an effective amount of the compound according to claim 11 or a pharmaceutically acceptable salt thereof.

50. A method for treating AIDS, which comprises administering an effective amount of the compound according to claim 9 or 10 or a pharmaceutically acceptable salt thereof.

51. A method for treating AIDS, which comprises administering an effective amount of the compound according to claim 11 or a pharmaceutically acceptable salt thereof.

52. A method for treating AIDS, which comprises administering an effective amount of a compound exhibiting an SH3 domain binding inhibitory activity or a pharmaceutically acceptable salt thereof.

53. A method for treating a viral disease, which comprises administering an effective amount of a compound exhibiting an SH3 domain binding inhibitory activity or a pharmaceutically acceptable salt thereof.

54-57. (canceled).

58. A method for inhibiting SH3 domain binding, which comprises administering an effective amount of the compound according to claim 10 or a pharmaceutically acceptable salt thereof.

59. The method for inhibiting SH3 domain binding according to claim 19, wherein said protein containing a proline-rich sequence is HIV-1Nef, p22-phox, p47-phox, Sam68, Sos 1, Dynamin, c-Cbl, ZO1, pX ORF I, LHDAg, NS5A, pORF3, ICP10, LMP2A, Tip or Tio.

60. The method for inhibiting SH3 domain binding according to any one of claims 14, 15 and 58, wherein said SH3 domain binding is an interaction between a protein containing an SH3 domain and a protein containing a proline-rich sequence.

61. The method for inhibiting SH3 domain binding according to claim 60, wherein the protein containing an SH3 domain and/or the protein containing a proline-rich sequence are/is virus-derived protein(s).

62. The method for inhibiting SH3 domain binding according to claim 61, wherein said virus-derived protein is a retrovirus-derived protein, a hepatitis virus-derived protein or a herpes virus-derived protein.

63. The method for inhibiting_SH3 domain binding according to claim 60, wherein said protein containing an SH3 domain is Src, Yes, Fgr, Hck, Lck, Abl, Fyn, Lyn, Blk, Yrk, Ras-GAP, PLCγ, PI3K, Tec, Txk/Rlk, Tsk/Emt/Itk, Btk, Crk, Grb2, Nck, Vav, STAT, Cortactin, p40-phox, p67-phox, p47-phox, a TCR signaling molecule (TCRsm), or a β chain or a γ chain of IL-2R.

64. The method for inhibiting SH3 domain binding according to claim 60, wherein said protein containing a proline-rich sequence is HIV-1Nef, p22-phox, p47-phox, Sam68, Sos1, Dynamin, c-Cbl, ZO1, pX ORF I, LHDAg, NS5A, pORF3, ICP10, LMP2A, Tip or Tio.

65. The method for inhibiting SH3 domain binding according to claim 60, wherein said interaction between a protein containing an SH3 domain and a protein containing a proline-rich sequence is an interaction between Grb2 and Sos1, Fyn and Sam68, Src and Sam68, PLCγ and Sam68, Grb2 and Sam68, Lyn and HIV-1Nef, Hck and HIV-1Nef, TCRsm and HIV-1Nef, p47-phox and p22-phox, p67-phox and p47-phox, Lyn and Dynamin, Cortactin and ZO1, Lyn and c-Cbl, a 0 chain or a y chain of IL-2R and pX ORF I, Grb2 and NS5A, Src and pORF3, Hck and pORF3, Fyn and pORF3, P13K and pORF3, PLCγ and pORF3, Grb2 and pORF3, Grb2 and ICP10, Lyn and LMP2A, Lck and Tip, Lyn and Tio, Hck and Tio, Lck and Tio, Src and Tio, Fyn and Tio, or Yes and Tio.

66. The method for inhibiting SH3 domain binding according to claim 60, wherein said interaction between a protein containing an SH3 domain and a protein containing a proline-rich sequence is an interaction between Grb2 and Sos1, Fyn and Sam68, Src and Sam68, PLCγ and Sam68, Grb2 and Sam68, Lyn and HIV-1Nef, or Cortactin and ZO1.

67. The method for inhibiting SH3 domain binding according to claim 63, wherein said protein containing a proline-rich sequence is HIV-1Nef, p22-phox, p47-phox, Sam68, Sos1, Dynamin, c-Cbl, ZO1, pX ORF I, LHDAg, NS5A, pORF3, ICP10, LMP2A, Tip or Tio.


SEQUENCE LISTING (1 sequence)

SEQ ID NO: 1 - DNA, 1228 bp, MPC1005
agcctgcatgtctaagtataagcaattataccgcgaaactgcgaatggctcattaaatcagttatcgtttatttgatagtaccttactacttggataaccgtggtaattctagagctaatacatgctaaaaatcccgacttacggagggatgtatttattagattaaaaaccaatgcccttcggggctccttggtgattcataataacttttcgaatcgcatggccttgcgccggcgatggttcattcaaatttctgccctatcaactttcgatggcagggtcttggcctgccatggttacaacgggtaacggagggttagggctcgaccccggagaaggagcctgagaaacggctactacatccaaggaaggcagcaggcgcgcaaattacccaatcccgattcggggaggtagtgacaataaatactgatacagggctctttcgggtcttgtaattggaatgagtacaatttaaatcccttaacgaggaacaattggagggcaagtctggtgccagcagccgcggtaattccagctccaatagcgtatattaaagttgttgcagttaaaaagctcgtagttgaaccttgggcctggctggccggtccgcctcaccgcgtgcactggttcggccgggcctttccctctggggatccccatgcccttcactgggtgtggtggggaaccaggacttttactgtgaaaaaattagagtgttcaaagcaggcctatgctcgaatacattagcatggaataatagaataggacgtgcggttctattttgttggtttctaggaccgccgtaatgattaatagggacagtcgggggcatcagtattcaattgtcagaggtgaaattcttggatttattgaagactaactactgcgaaagcatttgccaaggatgttttcattaatcaggaacgaaagttaggggatcgaagacgatcagataccgtcgtagtcttaaccataaactatgccgactagggatcggacgatgttattttttgactcgttcggcaccttacgagaaatcaaagtctttgggttctggggggagtatggtcgcaaggctgaaacttaaagaaattgacggaagggcaccaccaggagtggagcctgcggcttaatttgactcaacacggggaaactcaccaggtccagacacaatgaggattgacagattgagagctctttcttgattttgtgggtggtggtgcatgg